(19) United States
(12) Patent Application Publication
(10) Pub. No.: US 2019/0117572 A1
(43) Pub. Date: Apr. 25, 2019

(54) MICROFLUIDIC-FORMULATED LEUKOSOME COMPOSITIONS AND FABRICATION METHODS THEREFOR

(71) Applicant: The Methodist Hospital, Houston, TX (US)

(72) Inventors: Ennio Tasciotti, Houston, TX (US); Roberto Molinaro, Brookline, MA (US)

(21) Appl. No.: 15/915,055

(22) Filed: Mar. 7, 2018

Related U.S. Application Data
(63) Continuation of application No. PCT/US2017/018991, filed on Feb. 22, 2017.
(60) Provisional application No. 62/298,339, filed on Feb. 22, 2016.

(51) Int. Cl. A61K 9/127; C12N 15/113; A61K 51/12; A61K 49/00; A61K 49/18

(52) U.S. Cl. CPC A61K 9/1275; A61K 9/1273; A61K 9/1277; C12N 15/113; C12N 2320/32; A61K 49/0086; A61K 49/1812; C12N 2310/14; A61K 51/1237

(57) ABSTRACT

Disclosed are methods for designing and manufacturing biomimetic proteolipid nanovesicles using a microfluidic approach, and in particular, a NanoAssemblr®-based platform, which allows for the high-throughput, reproducible, and scalable production of nanoparticles, without affecting their pharmaceutical and biological properties. These nanovesicles, which are composed of synthetic phospholipids and cholesterol, enriched of leukocyte membranes, and surrounding an aqueous core, possess remarkable properties for targeting compounds of interest to particular mammalian cell and tissue types.

### CROSS-REFERENCE TO RELATED APPLICATIONS

The present application is a continuation-in-part application based on PCT Intl. Patent Appl. No. PCT/US2017/18991, filed Feb. 22, 2017 (pending; Atty. Dkt. No. 37182.194WO01); which claims priority to United States Provisional Patent Appl. No. 62/298,339, filed Feb. 22, 2016 (expired; Atty. Dkt. No. 37182.194PV01); the contents of each of which is specifically incorporated herein in its entirety by express reference thereto.

### STATEMENT REGARDING FEDERALLY SPONSORED RESEARCH OR DEVELOPMENT

Not Applicable.

### NAMES OF THE PARTIES TO A JOINT RESEARCH AGREEMENT

Not Applicable.

### BACKGROUND OF THE INVENTION

### Field of the Invention

The present invention relates generally to the field of medicine, and in particular, to methods for preparing nanovector-based drug delivery compositions. Disclosed are biomimetic proteolipid nanovesicles that possess remarkable properties for targeting compounds of interest to particular mammalian cells and tissue types. In particular embodiments, methods are disclosed for producing these vesicles that employ a microfluidics-based synthesis (including those employing the NanoAssemblr® platform), which allows for the high-throughput, reproducible, and scalable production of nanoparticles, without affecting their pharmaceutical and biological properties, are described. The versatility of a microfluidics-based approach makes it suitable for good manufacturing practice (GMP)-compliant manufacture of such biomimetic nanoparticles.

### Description of Related Art

A primary directive of nanotechnology is to develop drug delivery platforms that effectively reduce systemic toxicity of currently used drugs while retaining their pharmacological activity. To this purpose, several organic [lipids (Torchilin, 2014); polymers (Mitragotri et al., 2014)] and inorganic [silicon (Tasciotti et al., 2008), silica (Parodi et al., 2014); gold (Mura et al., 2013); and iron oxide (Kudgus et al., 2014) materials have been manipulated at the micro- and nano-scale to synthesize drug carriers. In the development of such materials bio-inspired approaches have emerged to face the extraordinary ability of the human body to recognize, label, sequester, and clear foreign objects (Luk and Zhang, 2015). By combining the typical properties of conventional synthetic nanoparticles (Ferrari, 2005; Torchilin, 2014) with natural (Hu et al., 2011; Hu et al., 2015; Parodi et al., 2013) or biomimetic (Hammer et al., 2008; Doshi et al., 2012) materials, such strategies revolutionized the concept of drug delivery in nanomedicine (Blanco et al., 2015).

In this scenario, bottom-up approaches opened the door to the synthesis of bio-inspired delivery systems through surface functionalization with targeting molecules that mimic plasma membrane receptors of specialized cells. Pioneering in this field was the design and development of Leuko-polymersomes that reconstituted two important leukocyte-derived adhesion molecules (analogs of P-selectin glycoprotein ligand 1 (PSGL-1) and leukocyte function-associated antigen (LFA) 1 receptors) on the surface of polymersomes (Robbins et al., 2010). The presence of these two receptor mimetics was shown to ensure avid and highly selective binding to the inflamed endothelium both in vitro and in vivo, thus suggesting that the contribution of both pathways is fundamental to mimic the leukocyte targeting properties (Robbins et al., 2010).

Using a similar approach, platelet-like nanoparticles displaying surface-binding, site-selective adhesion, and aggregation properties effectively mimicking platelets and their hemostatic functions, were synthesized (Anselmo et al., 2014). Although bottom-up approaches have the great advantage of providing superior physicochemical control over the final formulation, current chemical conjugation methods remain inadequate to reproduce the complexity of the cellular membrane on the surface of nanocarriers (Luk and Zhang, 2015). In fact, the contemporary addition of distinctive elements on the surface of drug carriers requires complex synthesis and purification protocols, whose complexity increases as a function of the number of different components.

To further bridge the gap between synthetic nanoparticles and biological materials, top-down procedures were developed. In this scenario, the use of plasma membranes derived from various cell types [i.e., red blood cells (Hu et al., 2011); platelets (Hu et al., 2015); leukocytes (Parodi et al., 2013); stem cells (Toledano-Furman et al., 2013)] to coat the surface of synthetic particles (Parodi et al., 2013) or used as carriers per se [cell ghosts for instance (Hu et al., 2011; 2015)], permitted the faithful recapitulation of the cellular biological complexity on the carrier's surface. The resulting biomimetic coating offers a one-step solution to simultaneously bestow particles with multiple bioactive functions including evasion of the mononuclear phagocytic system (MPS) and negotiation across various biological barriers (Yoo et al., 2011; Alvarez-Lorenzo and Concheiro, 2013).

The advancement of nanotechnology toward more sophisticated bio-inspired approaches has highlighted the gap between the advantages of biomimetic and bio-hybrid platforms and the availability of manufacturing processes to scale-up their production. Though recent works have reported the advantages of transferring biological features from cells to synthetic nanoparticles for drug delivery purposes, a standardizable, batch-to-batch consistent, scalable, and high-throughput assembly method is required to further develop these platforms. Microfluidics has offered a robust tool for the controlled synthesis of nanoparticles in a versatile and reproducible approach. In this study, the incorporation of membrane proteins within the bilayer of biomimetic nanovesicles (Leukosomes) using a microfluidic-based platform is demonstrated. The physical, pharmaceutical and biological properties of microfluidic-formulated Leukosomes (called NA-Leuko) are also characterized. NA-Leuko showed extended shelf-life and retention of the biological functions of donor cells (i.e., macrophage avoidance and targeting of inflamed vasculature). The NA approach represents a universal, versatile, robust, and scalable tool, which has been extensively used for the assembly of lipid nanoparticles and adapted here for the manufacturing of biomimetic nanovesicles.

### DEFICIENCIES IN THE PRIOR ART

Unfortunately, top-down approaches to the synthesis of nanovectors have their own limitations, such as issues in the control of physical parameters (i.e., size homogeneity) of the final formulation, poor control of the encapsulation and retention of chemically different molecules (i.e., hydrophilic, amphiphilic, and lipophilic small drugs), as well as difficulties involving the absence of a standardized protocol for preparation, contamination, and storage (Gutiérrez Millán et al., 2012; Millan et al., 2004). A more reliable, more stable, and more reproducible method of synthesis is therefor desirable.

### BRIEF SUMMARY OF THE INVENTION

The present invention overcomes these and other limitations inherent in the prior art by providing methods for the synthesis of biomimetic nanovesicles, termed leukosomes, which employ proteins derived from leukocyte plasma membranes integrated into a synthetic biocompatible phospholipid bilayer as a new drug delivery platform. In particular embodiments, a microfluidics-based synthesis is disclosed for preparation of populations of biomimetic nanovesicles having desired biochemical properties. The leukosomes synthesized and described herein provide improved drug delivery vehicles, which combine the cell properties of leukocytes and the drug-delivery features of liposomes to produce a highly-desirable method of drug delivery to mammalian cells and tissues.

In particular embodiments, the biomimetic nanovesicles of the present disclosure have been employed to provide combinational therapy of therapeutic molecules such as siRNAs treat one or more forms of inflammation, or, when combined with one or more chemotherapeutics, to treat one or more types of cancer in an affected individual. These nanovector-based drug delivery compositions improve the accumulation of conventional drugs in selected mammalian tissues, and achieve better therapeutic effect over the available conventional therapies.

The present disclosure provides in one aspect, biomimetic nanovesicles that improve stability and loading doses for mammalian administration.

These nanovesicles can be used to preferentially target particular cell types, reduce phlogosis in a localized model of inflammation, while retaining both the versatility and physicochmiecal properties typical of conventional nanovesicle formulations.

Chemotherapeutic Methods and Use

An important aspect of the present disclosure concerns methods for using the disclosed drug delivery formulations for treating or ameliorating the symptoms of one or more forms of cancer, including, for example, a metastatic cancer, such as melanoma metastasis to the mammalian lung. Such methods generally involve administering to a mammal (and in particular, to a human in need thereof), one or more of the disclosed drug delivery systems comprising at least a first anti-inflammatory and/or an anticancer composition, in an amount and for a time sufficient to treat (or, alternatively ameliorate one or more symptoms of) the identified inflammation and/or cancer in an affected mammal.

In certain embodiments, the nanovesicle formulations described herein may be provided to the animal in a single treatment modality (either as a single administration, or alternatively, in multiple administrations over a period of from several hours (hrs) to several days (or even several weeks or several months) as needed to treat the particular disease, disorder, dysfunction, or abnormal condition. Alternatively, in some embodiments, it may be desirable to continue the treatment, or to include it in combination with one or more additional modes of therapy, for a period of several months or longer. In other embodiments, it may be desirable to provide the therapy in combination with one or more conventional treatment regimens.

The present disclosure also provides for the use of one or more of the disclosed nanovesicle drug delivery compositions in the manufacture of a medicament for therapy and/or for the amelioration of one or more symptoms of inflammation and/or cancer, and particularly for use in the manufacture of a medicament for treating and/or ameliorating one or more symptoms of a mammalian inflammation and/or cancer, including, for example human diseases and disorders.

The present invention also provides for the use of one or more of the disclosed drug delivery nanovesicle formulations in the manufacture of a medicament for the treatment of a disease or disorder in a mammal, and in particular, for the treatment of one or more human diseases such as inflammation and/or cellular hyperproliferation (i.e., cancer).

Therapeutic Kits

Therapeutic kits including one or more of the disclosed nanovesicle drug delivery compositions and instructions for using the kit in a particular treatment modality also represent preferred aspects of the present disclosure. These kits may further optionally include one or more additional therapeutic compounds, one or more diagnostic reagents, or any combination thereof.

The kits of the invention may be packaged for commercial distribution, and may further optionally include one or more delivery devices adapted to deliver the micro/nano composite drug delivery composition(s) to an animal (e.g., syringes, injectables, and the like). Such kits typically include at least one vial, test tube, flask, bottle, syringe, or other container, into which the micro/nano composite drug delivery composition(s) may be placed, and preferably suitably aliquotted. Where a second pharmaceutical is also provided, the kit may also contain a second distinct container into which this second composition may be placed. Alternatively, the plurality of leukosomes disclosed herein may be prepared in a single mixture, such as a suspension or solution, and may be packaged in a single container, such as a vial, flask, syringe, catheter, cannula, bottle, or other suitable single container.

The kits of the present invention may also typically include a retention mechanism adapted to contain or retain the vial(s) or other container(s) in close confinement for commercial sale, such as, e.g., injection or blow-molded plastic containers into which the desired vial(s) or other container(s) may be retained to minimize or prevent breakage, exposure to sunlight, or other undesirable factors, or to permit ready use of the composition(s) included within the kit.

Pharmaceutical Formulations

In certain embodiments, the present invention concerns formulation of one or more chemotherapeutic and/or diagnostic compounds in a pharmaceutically acceptable formulation of the leukosome compositions disclosed herein for administration to one or more cells or tissues of an animal, either alone, or in combination with one or more other modalities of diagnosis, prophylaxis, and/or therapy. The formulation of pharmaceutically acceptable excipients and carrier solutions is well known to those of ordinary skill in the art, as is the development of suitable dosing and treatment regimens for using the particular compositions described herein in a variety of treatment regimens.

In certain circumstances it will be desirable to deliver the disclosed chemotherapeutic compositions in suitably-formulated pharmaceutical vehicles by one or more standard delivery devices, including, without limitation, subcutaneously, parenterally, intravenously, intramuscularly, intrathecally, orally, intraperitoneally, transdermally, topically, by oral or nasal inhalation, or by direct injection to one or more cells, tissues, or organs within or about the body of an animal.

The methods of administration may also include those modalities as described in U.S. Pat. Nos. 5,543,158; 5,641,515, and 5,399,363, each of which is specifically incorporated herein in its entirety by express reference thereto. Solutions of the active compounds as freebase or pharmacologically acceptable salts may be prepared in sterile water, and may be suitably mixed with one or more surfactants, such as hydroxypropylcellulose. Dispersions may also be prepared in glycerol, liquid polyethylene glycols, oils, or mixtures thereof. Under ordinary conditions of storage and use, these preparations contain a preservative to prevent the growth of microorganisms.

For administration of an injectable aqueous solution, without limitation, the solution may be suitably buffered, if necessary, and the liquid diluent first rendered isotonic with sufficient saline or glucose.

These particular aqueous solutions are especially suitable for intravenous, intramuscular, subcutaneous, transdermal, subdermal, and/or intraperitoneal administration. In this regard, the leukosome compositions of the present invention may be formulated in one or more pharmaceutically acceptable vehicles, including for example sterile aqueous media, buffers, diluents, etc. For example, a given dosage of active ingredient(s) may be dissolved in a particular volume of an isotonic solution (e.g., an isotonic NaCl-based solution), and then injected at the proposed site of administration, or further diluted in a vehicle suitable for intravenous infusion (see, e.g., “REMINGTON'S PHARMACEUTICAL SCIENCES” 15^{th} Ed., pp. 1035-1038 and 1570-1580). While some variation in dosage will necessarily occur depending on the condition of the subject being treated, the extent of the treatment, and the site of administration, the person responsible for administration will nevertheless be able to determine the correct dosing regimens appropriate for the individual subject using ordinary knowledge in the medical and pharmaceutical arts.

Sterile injectable leukosome compositions may be prepared by incorporating the disclosed chemotherapeutic delivery system formulations in the required amount in the appropriate solvent with several of the other ingredients enumerated above, as required, followed by filtered sterilization. Generally, dispersions can be prepared by incorporating the selected sterilized active ingredient(s) into a sterile vehicle that contains the basic dispersion medium and the required other ingredients from those enumerated above. The compositions disclosed herein may also be formulated in a neutral or salt form.

Pharmaceutically acceptable salts include the acid addition salts (formed with the free amino groups of the protein), and which are formed with inorganic acids such as, without limitation, hydrochloric or phosphoric acids, or organic acids such as, without limitation, acetic, oxalic, tartaric, mandelic, and the like. Salts formed with the free carboxyl groups can also be derived from inorganic bases such as, without limitation, sodium, potassium, ammonium, calcium, or ferric hydroxides, and such organic bases as isopropylamine, trimethylamine, histidine, procaine, and the like. Upon formulation, solutions will be administered in a manner compatible with the dosage formulation, and in such amount as is effective for the intended application. The formulations are readily administered in a variety of dosage forms such as injectable solutions, topical preparations, oral formulations, including sustain-release capsules, hydrogels, colloids, viscous gels, transdermal reagents, intranasal and inhalation formulations, and the like.

The amount, dosage regimen, formulation, and administration of chemotherapeutics disclosed herein will be within the purview of the ordinary-skilled artisan having benefit of the present teaching. It is likely, however, that the administration of a therapeutically-effective (i.e., a pharmaceutically-effective, chemotherapeutically-effective, or an anticancer-effective) amount of the disclosed leukosome drug delivery formulations may be achieved by a single administration, such as, without limitation, a single injection of a sufficient quantity of the delivered agent to provide the desired benefit to the patient undergoing such a procedure. Alternatively, in other circumstances, it may be desirable to provide multiple, or successive administrations of leukosome compositions disclosed herein, over relatively short or even relatively prolonged periods, as may be determined by the medical practitioner overseeing the administration of such compositions to the selected individual.

Typically, the leukosome compositions described herein will contain at least a chemotherapeutically-effective amount of a first active agent. Preferably, the formulation may contain at least about 0.001% of each active ingredient, preferably at least about 0.01% of the active ingredient, although the percentage of the active ingredient(s) may, of course, be varied, and may conveniently be present in amounts from about 0.01 to about 90 weight % or volume %, or from about 0.1 to about 80 weight % or volume %, or more preferably, from about 0.2 to about 60 weight % or volume %, based upon the total formulation. Naturally, the amount of active compound(s) in each composition may be prepared in such a way that a suitable dosage will be obtained in any given unit dose of the compound. Factors such as solubility, bioavailability, biological t_{1/2}, route of administration, product shelf life, as well as other pharmacological considerations will be contemplated by one of ordinary skill in the art of preparing such pharmaceutical formulations, and as such, a variety of dosages and treatment regimens may be desirable.

Administration of the leukosome compositions disclosed herein may be administered by any effective method, including, without limitation, by parenteral, intravenous, intramuscular, or even intraperitoneal administration as described, for example, in U.S. Pat. Nos. 5,543,158; 5,641,515; and 5,399,363 (each of which is specifically incorporated herein in its entirety by express reference thereto). Solutions of the active compounds as free-base or pharmacologically acceptable salts may be prepared in water suitably mixed with a surfactant, such as hydroxypropylcellulose, or other similar fashion. The pharmaceutical forms adapted for injectable administration include sterile aqueous solutions or dispersions, and sterile powders for the extemporaneous preparation of sterile injectable solutions or dispersions including without limitation those described in U.S. Pat. No. 5,466,468 (specifically incorporated herein in its entirety by express reference thereto). In all cases, the form must be sterile and must be fluid to the extent that easy syringability exists. It must be at least sufficiently stable under the conditions of manufacture and storage, and must be preserved against the contaminating action of microorganisms, such as viruses, bacteria, fungi, and such like.

Exemplary carrier(s) may include, for example, a solvent or dispersion medium, including, without limitation, water, ethanol, polyol (e.g., glycerol, propylene glycol, and liquid polyethylene glycol, and the like, or a combination thereof), one or more vegetable oils, or any combination thereof, although additional pharmaceutically-acceptable components may be included.

Proper fluidity of the pharmaceutical formulations disclosed herein may be maintained, for example, by the use of a coating, such as e.g., a lecithin, by the maintenance of the required particle size in the case of dispersion, by the use of a surfactant, or any combination of these techniques. The inhibition or prevention of the action of microorganisms can be brought about by one or more antibacterial or antifungal agents, for example, without limitation, a paraben, chlorobutanol, phenol, sorbic acid, thimerosal, or the like. In many cases, it will be preferable to include an isotonic agent, for example, without limitation, one or more sugars or sodium chloride, or any combination thereof. Prolonged absorption of the injectable compositions can be brought about by the use in the compositions of agents delaying absorption, for example without limitation, aluminum monostearate, gelatin, or a combination thereof.

While systemic administration is contemplated to be effective in many embodiments of the invention, it is also contemplated that formulations disclosed herein be suitable for direct injection into one or more organs, tissues, or cell types in the body. Direct organ administration of the disclosed leukosome compositions may be conducted using suitable means, including those known to those of ordinary skill in the oncological arts.

The pharmaceutical formulations of the leukosome compositions disclosed herein are not in any way limited to use only in humans, or even to primates, or mammals. In certain embodiments, the methods and drug delivery formulations disclosed herein may be employed using avian, amphibian, reptilian, or other animal species. In preferred embodiments, however, the drug delivery compositions of the present invention are preferably formulated for administration to a mammal, and in particular, to humans, as part of an oncology regimen for treating one or more cancers. The leukosome compositions disclosed herein may also be acceptable for veterinary administration, including, without limitation, to selected livestock, exotic or domesticated animals, companion animals (including pets and such like), non-human primates, as well as zoological or otherwise captive specimens, and the like.

### BRIEF DESCRIPTION OF THE DRAWINGS

The following drawings form part of the present specification and are included to demonstrate certain aspects of the present invention. The patent or application file contains at least one drawing executed in color. Copies of this patent or patent application publication with color drawing(s) will be provided by the Office upon request and payment of the necessary fee.

For promoting an understanding of the principles of the invention, reference will now be made to the embodiments, or examples, illustrated in the drawings and specific language will be used to describe the same. It will, nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates.

The invention may be better understood by reference to the following description taken in conjunction with the accompanying drawings, in which like reference numerals identify like elements, and in which:

FIG. 1A, FIG. 1B, FIG. 1C, FIG. 1D, FIG. 1E, FIG. 1F, and FIG. 1G show the continuous-based process for the assembly of biomimetic nanovesicles using NanoAssemblr® platform. FIG. 1A) Schematic representation of Leukosomes microfluidic synthesis (not to scale). FIG. 1B) Dynamic Light Scattering (DLS) analysis shows average diameter, polydispersity (PDI) index, and FIG. 1C) zeta potential of Liposomes (Lipo) and NA-Leuko after membrane protein incorporation at 1:300, 1:100, and 1:50 protein to lipid ratios. FIG. 1D) Deformability index shows vesicles' flexibility following to membrane proteins incorporation at 1:300, 1:100, and 1:50 protein to lipid ratios, and identifies the 1:50 ratio as the highest level of protein incorporation. Results represent the average of at least three different batches of particles±standard deviation. FIG. 1E) High-magnification cryoEM analysis of 1:50 NA-Leuko reveals spherical shape, unilamellar vesicles, and validates DLS analysis. FIG. 1F) Liposome and FIG. 1G) NA-Leuko surface profile using atomic force microscopy (AFM) analysis. **p<0.1; ***p<0.01; ****p<0.001;

FIG. 2A, FIG. 2B, and FIG. 2C show the physical stability of biomimetic NA-Leuko. Transmission (AT %) and backscattering (ABS %) profiles of liposomes (FIG. 2A) and NA-Leuko (FIG. 2B) using Turbiscan Lab®. The analysis was performed at 20° C. Data are reported as a function of time (0-1 hr) and sample height (from 2 to 15 mm). FIG. 2C: DLS analysis of NA-Leuko stored in solution at 4° C. up to 24 days reveals no significant change in size and homogeneity;

FIG. 3A, FIG. 3B, FIG. 3C, FIG. 3D, FIG. 3E, and FIG. 3F show the orientation of membrane proteins within the bilayer of NanoAssemblr-derived leukosomes (NA-Leuko). Flow cytometry analysis performed on liposomes and NA-Leuko revealed FIG. 3A) the presence and correct orientation of LFA-1 and CD47 on NA-Leuko surface, FIG. 3B) the absence of signal from intracellular domains CD3z of membrane proteins, and FIG. 3C) the presence of glycosylated domains on NA-Leuko surface and self-assembled membrane proteins (positive control). FIG. 3D) Coarse-grained model represents 3D structure of the integral protein MHC I in the orientation OUT, i.e., glycosylated domain oriented outside NA-Leuko bilayer. FIG. 3E) and IN, glycosylated domain oriented inside NA-Leuko bilayer, respectively. FIG. 3F) Total Energy calculated as difference between the energies relative to the outside and inside orientation, identified as OUT and IN, respectively, of the glycosylated domain of the integral protein MHC I. The energy values are reported in kJ mol⁻¹, indicated in a range between −4649 and −4653×10³ kJ mol⁻¹. **p<0.1; ***p<0.01; ****p<0.001;

FIG. 4A, FIG. 4B, FIG. 4C, FIG. 4D, FIG. 4E, and FIG. 4F show the in vitro and in vivo biological properties of NA-Leuko. In vitro uptake studies of control liposomes and murine NA-Leuko following incubation with J774 macrophages using FIG. 4A: confocal microscopy (24 hr incubation) and FIG. 4B: flow cytometry (6 and 24 hr incubation). Both the techniques showed reduced phagocytosis for the biomimetic nanovesicles. Scale bar 25 μm. GREEN: cell membranes stained with FITC-labeled wheat germ agglutinin; BLUE: nuclei stained with DAPI; RED: particles labeled with rhodamine. FIG. 4C: Dynamic flow chamber experiments study the adhesion of control liposomes and NA-Leuko towards human umbilical vein endothelial cells (HUVEC). FIG. 4D) In vivo inflammatory targeting of NA-Leuko in a localized (i.e., ear) inflammation model (n=8-12 images from 3 mice, graphs represent the means+/−s.e.m). Representative IVM images show preferential accumulation of NA-Leuko in inflamed ears at 1 and 24 h with minimal accumulation observed in healthy ears. Vessels are shown in green and NA-Leuko in red, scale bar 200 μm. FIG. 4E and FIG. 4F: Quantification revealed an 8- and 13-fold increased accumulation of NA-Leuko into the inflamed ear compared to healthy ears at both 1 and 24 hr after injection, respectively. *p<0.5; **p<0.1; ****p<0.001;

FIG. 5A, FIG. 5B, FIG. 5C, FIG. 5D, FIG. 5E, and FIG. 5F show low-magnification cryoEM images and size distribution traces of 1:300, 1:100, and 1:50 NA-Leuko reveal homogeneity in size. Scale bar 100 nm;

FIG. 6A, FIG. 6B, and FIG. 6C show reproducibility of the assembly protocol. Size distribution traces of NA-Leuko assembled using NanoAssemblr™ Benchtop platform by 3 independent operators are reported. The inter-operator variability is minor—less than 2.5%, thus indicating high reproducibility of the assembly protocols;

FIG. 7A, FIG. 7B, FIG. 7C, and FIG. 7D show the evaluation of membrane proteins' incorporation within NA-Leuko. The percentage of membrane proteins associated to NA-Leuko is reported. To avoid the interference of lipids with the Bradford assay, the amount of proteins incorporated into the NA-Leuko has been calculated indirectly as the difference between the amount of protein initially added to the formulation minus the amount not associated to NA-Leuko after assembly. Briefly, after assembly (FIG. 7A), NA-Leuko were ultracentrifuged (FIG. 7B) to separate the nanovesicles (yellow pellet) from the unincorporated material (lipids and membrane proteins). Next, the supernatant containing the membrane proteins was dialyzed (FIG. 7C) to eliminate unincorporated lipids and analyzed through Bradford assay (FIG. 7D) to quantify the amount of membrane proteins not associated to NA-Leuko;

FIG. 8 shows the theoretical calculation of NA-Leuko compared to TLE-Leuko. The NanoAssemblr-based approach allowed for 14-fold increase of membrane proteins into the NA-Leuko bilayer. For what regards the amount of membrane proteins per μm2 of surface area, a 22.7-fold increase for NA-Leuko compared to TLE-Leuko can be observed. The number of vesicles per gram of lipids produced using the microfluidic approach is 2.18-fold higher, thus revealing a higher production efficiency. The values reported in the graphs were normalized for the gram of lipids used for nanovesicles' assembly;

FIG. 9A, FIG. 9B, FIG. 9C, FIG. 9D, and FIG. 9E show Atomic Force Microscopy (AFM) analysis of control liposomes and NA-Leuko confirms DLS results and shows different viscoelastic and mechanical properties for the two formulations;

FIG. 10 shows profiles of Turbiscan Stability Index (TSI) of liposomes and NA-Leuko at 20° C. and 37° C. obtained by using Turbiscan Lab®;

FIG. 11A and FIG. 11B show flow cytometry analysis shows the absence of FIG. 11A) mitochondrial and nuclear contaminants in NA-Leuko formulation; FIG. 11B) Control liposomes and NA-Leuko have been incubated with DAPI to detect the presence of genetic material in the formulation;

FIG. 12 show the variation of Total Energy relative to the outside and inside orientation, identified as OUT and IN, respectively, of the glycosylated domain of the integral protein MEW I. The energy values are reported in kJ mol⁻¹, indicated in a range between −4664 and −4640×0³ kJ mol⁻¹, and plotted versus time, expressed in picoseconds (psec);

FIG. 13A and FIG. 13B show Lennard-Jones potential for the glycosylated domain oriented OUT vs IN was calculated. Time course (upper image) and end-point (lower image) analyses reveal that orientation OUT is more stable (i.e., lower energy) compared to orientation IN. The energy values are reported in kJ mol⁻¹ and are plotted versus time, expressed in picoseconds (psec);

FIG. 14A and FIG. 14B show Coulomb potential for the glycosylated domain oriented OUT vs IN was calculated. Time course (FIG. 14A) and end-point (FIG. 14B) analyses reveal that orientation OUT is more stable (i.e., lower energy) compared to orientation IN. The energy values are reported in kJ mol⁻¹ and are plotted versus time, expressed in picoseconds (psec); and

FIG. 15 shows proteins identified on Leukosomes. Proteins identified on leukosomes have been analyzed using STRING v.10.0 (string-db.org) to create proteins' functional association networks. The figure shows all proteins identified on leukosomes. Functional enrichments in the protein network are highlighted: red proteins are associated to the plasma membrane and to membrane-bounded vesicle (false discovery rate 1.2×10⁻²⁴), proteins indicated by arrows are connected to the KEGG Pathway Leukocyte Transendothelial Migration (false discovery rate 2.55×10⁻⁰⁷).

### DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would be a routine undertaking for those of ordinary skill in the art having the benefit of this disclosure.

Pharmaceutical Formulations

In certain embodiments, the present invention concerns nanovesicle compositions prepared in pharmaceutically-acceptable formulations for delivery to one or more cells or tissues of an animal, either alone, or in combination with one or more other modalities of diagnosis, prophylaxis and/or therapy. The formulation of pharmaceutically acceptable excipients and carrier solutions is well known to those of ordinary skill in the art, as is the development of suitable surgical implantation methods for using the particular membrane compositions described herein in a variety of treatment regimens, and particularly those involving bone regrowth.

Sterile injectable formulations may be prepared by incorporating the disclosed leukosome-based drug delivery compositions in the required amount in the appropriate solvent with several of the other ingredients enumerated above, as required, followed by filtered sterilization. Generally, dispersions can be prepared by incorporating the selected sterilized active ingredient(s) into a sterile vehicle that contains the basic dispersion medium and the required other ingredients from those enumerated above. The leukosome-based drug delivery compositions disclosed herein may also be formulated in solutions comprising a neutral or salt form to maintain the integrity of the vesicles prior to administration.

Pharmaceutically acceptable salts include the acid addition salts (formed with the free amino groups of the protein), and which are formed with inorganic acids such as, without limitation, hydrochloric or phosphoric acids, or organic acids such as, without limitation, acetic, oxalic, tartaric, mandelic, and the like. Salts formed with the free carboxyl groups can also be derived from inorganic bases such as, without limitation, sodium, potassium, ammonium, calcium, or ferric hydroxides, and such organic bases as isopropylamine, trimethylamine, histidine, procaine, and the like. Upon formulation, solutions will be administered in a manner compatible with the dosage formulation, and in such amount as is effective for the intended application. The formulations are readily administered in a variety of dosage forms such as injectable solutions, topical preparations, oral formulations, including sustain-release capsules, hydrogels, colloids, viscous gels, transdermal reagents, intranasal and inhalation formulations, and the like.

The amount, implantation regimen, formulation, and preparation of the leukosome-based drug delivery compositions disclosed herein will be within the purview of the ordinary-skilled artisan having benefit of the present teaching. It is likely, however, that the administration of a particular leukosome composition may be achieved by a single administration to provide the desired benefit to the patient undergoing such a procedure. Alternatively, in some circumstances, it may be desirable to provide multiple, or successive administrations of the leukosome-based agents, either over a relatively short, or even a relatively prolonged period, as may be determined by the medical practitioner overseeing the individual undergoing treatment.

The leukosome-based drug delivery compositions disclosed herein are not in any way limited to use only in humans, or even to primates, or mammals. In certain embodiments, the methods and leukosome-based drug delivery compositions disclosed herein may be employed in the treatment of avian, amphibian, reptilian, and/or other animal species, and may be formulated for veterinary surgical use, including, without limitation, for administration to selected livestock, exotic or domesticated animals, companion animals (including pets and such like), non-human primates, as well as zoological or otherwise captive specimens, and such like.

Compositions for the Preparation of Medicaments

Another important aspect of the present invention concerns methods for using the disclosed leukosome compositions (as well as formulations including them) in the preparation of medicaments for treating and/or ameliorating one or more symptoms of one or more diseases, dysfunctions, abnormal conditions, or disorders in an animal, including, for example, vertebrate mammals.

Such use generally involves administration to the mammal in need thereof one or more of the disclosed leukosome compositions, in an amount and for a time sufficient to treat or ameliorate one or more symptoms of an injury, defect, or disease in an affected mammal.

Pharmaceutical formulations including one or more of the disclosed leukosome compositions also form part of the present invention, and particularly those compositions that further include at least a first pharmaceutically-acceptable excipient for use in the therapy and/or amelioration of one or more symptoms of disease, defect, abnormal condition, or trauma in an affected mammal.

### Exemplary Definitions

In accordance with the present invention, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from natural sources, chemically synthesized, modified, or otherwise prepared or synthesized in whole or in part by the hand of man.

Unless defined otherwise, all technical and scientific terms used herein have the same meaning as commonly understood by one of ordinary skill in the art to which this invention belongs. The following references provide one of skill with a general definition of many of the terms used in this invention: Dictionary of Biochemistry and Molecular Biology, (2^{nd} Ed.) J. Stenesh (Ed.), Wiley-Interscience (1989); Dictionary of Microbiology and Molecular Biology (3^{rd} Ed.), P. Singleton and D. Sainsbury (Eds.), Wiley-Interscience (2007); Chambers Dictionary of Science and Technology (2^{nd} Ed.), P. Walker (Ed.), Chambers (2007); Glossary of Genetics (5^{th} Ed.), R. Rieger et al. (Eds.), Springer-Verlag (1991); and The HarperCollins Dictionary of Biology, W. G. Hale and J. P. Margham, (Eds.), HarperCollins (1991).

Although any methods and compositions similar or equivalent to those described herein can be used in the practice or testing of the present invention, the preferred methods, and compositions are described herein. For purposes of the present invention, the following terms are defined below for sake of clarity and ease of reference:

In accordance with long standing patent law convention, the words “a” and “an,” when used in this application, including the claims, denote “one or more.”

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

As used herein, “bioactive” shall include a quality of a material such that the material has an osteointegrative potential, or in other words the ability to bond with bone. Generally, materials that are bioactive develop an adherent interface with tissues that resist substantial mechanical forces.

As used herein, a “biocompatible” material is a synthetic or natural material used to replace part of a living system or to function in intimate contact with living tissue. Biocompatible materials are intended to interface with biological systems to evaluate, treat, augment, or replace any tissue, organ, or function of the body. The biocompatible material has the ability to perform with an appropriate host response in a specific application and does not have toxic or injurious effects on biological systems. One example of a biocompatible material can be a biocompatible ceramic.

The term “biologically-functional equivalent” is well understood in the art, and is further defined in detail herein. Accordingly, sequences that have about 85% to about 90%; or more preferably, about 91% to about 95%; or even more preferably, about 96% to about 99%; of nucleotides that are identical or functionally-equivalent to one or more of the nucleotide sequences provided herein are particularly contemplated to be useful in the practice of the methods and compositions set forth in the instant application.

As used herein, “biomimetic” shall mean a resemblance of a synthesized material to a substance that occurs naturally in a human body and which is not rejected by (e.g., does not cause an adverse reaction in) the human body.

As used herein, the term “buffer” includes one or more compositions, or aqueous solutions thereof, that resist fluctuation in the pH when an acid or an alkali is added to the solution or composition that includes the buffer. This resistance to pH change is due to the buffering properties of such solutions, and may be a function of one or more specific compounds included in the composition. Thus, solutions or other compositions exhibiting buffering activity are referred to as buffers or buffer solutions. Buffers generally do not have an unlimited ability to maintain the pH of a solution or composition; rather, they are typically able to maintain the pH within certain ranges, for example from a pH of about 5 to 7.

As used herein, the term “carrier” is intended to include any solvent(s), dispersion medium, coating(s), diluent(s), buffer(s), isotonic agent(s), solution(s), suspension(s), colloid(s), inert (s), or such like, or a combination thereof that is pharmaceutically acceptable for administration to the relevant animal or acceptable for a therapeutic or diagnostic purpose, as applicable.

As used herein, “chondrocyte” shall mean a differentiated cell responsible for secretion of extracellular matrix of cartilage. Preferably, the cells are from a compatible human donor. More preferably, the cells are from the patient (i.e., autologous cells).

As used herein, the term “DNA segment” refers to a DNA molecule that has been isolated free of total genomic DNA of a particular species. Therefore, a DNA segment obtained from a biological sample using one of the compositions disclosed herein refers to one or more DNA segments that have been isolated away from, or purified free from, total genomic DNA of the particular species from which they are obtained. Included within the term “DNA segment,” are DNA segments and smaller fragments of such segments, as well as recombinant vectors, including, for example, plasmids, cosmids, phage, viruses, and the like.

The term “effective amount,” as used herein, refers to an amount that is capable of treating or ameliorating a disease or condition or otherwise capable of producing an intended therapeutic effect.

As used herein, “fibroblast” shall mean a cell of connective tissue that secretes proteins and molecular collagen including fibrillar procollagen, fibronectin and collagenase, from which an extracellular fibrillar matrix of connective tissue may be formed. Fibroblasts synthesize and maintain the extracellular matrix of many tissues, including but not limited to connective tissue. The fibroblast cell may be mesodermally derived, and secrete proteins and molecular collagen including fibrillar procollagen, fibronectin and collagenase, from which an extracellular fibrillar matrix of connective tissue may be formed. A “fibroblast-like cell” means a cell that shares certain characteristics with a fibroblast (such as expression of certain proteins).

The terms “for example” or “e.g.,” as used herein, are used merely by way of example, without limitation intended, and should not be construed as referring only those items explicitly enumerated in the specification.

As used herein, “hard tissue” is intended to include mineralized tissues, such as bone, teeth, and cartilage. Mineralized tissues are biological tissues that incorporate minerals into soft matrices.

As used herein, a “heterologous” sequence is defined in relation to a predetermined, reference sequence, such as, a polynucleotide or a polypeptide sequence. For example, with respect to a structural gene sequence, a heterologous promoter is defined as a promoter which does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements.

As used herein, “homologous” means, when referring to polynucleotides, sequences that have the same essential nucleotide sequence, despite arising from different origins. Typically, homologous nucleic acid sequences are derived from closely related genes or organisms possessing one or more substantially similar genomic sequences. By contrast, an “analogous” polynucleotide is one that shares the same function with a polynucleotide from a different species or organism, but may have a significantly different primary nucleotide sequence that encodes one or more proteins or polypeptides that accomplish similar functions or possess similar biological activity. Analogous polynucleotides may often be derived from two or more organisms that are not closely related (e.g., either genetically or phylogenetically).

As used herein, the term “homology” refers to a degree of complementarity between two or more polynucleotide or polypeptide sequences. The word “identity” may substitute for the word “homology” when a first nucleic acid or amino acid sequence has the exact same primary sequence as a second nucleic acid or amino acid sequence. Sequence homology and sequence identity can be determined by analyzing two or more sequences using algorithms and computer programs known in the art. Such methods may be used to assess whether a given sequence is identical or homologous to another selected sequence.

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of ordinary skill) or by visual inspection.

As used herein, “implantable” or “suitable for implantation” means surgically appropriate for insertion into the body of a host, e.g., biocompatible, or having the desired design and physical properties.

As used herein, the phrase “in need of treatment” refers to a judgment made by a caregiver such as a physician or veterinarian that a patient requires (or will benefit in one or more ways) from treatment. Such judgment may be made based on a variety of factors that are in the realm of a caregiver's expertise, and may include the knowledge that the patient is ill as the result of a disease state that is treatable by one or more compound or pharmaceutical compositions such as those set forth herein.

The phrases “isolated” or “biologically pure” refer to material that is substantially, or essentially, free from components that normally accompany the material as it is found in its native state.

As used herein, the term “kit” may be used to describe variations of the portable, self-contained enclosure that includes at least one set of reagents, components, or pharmaceutically-formulated compositions to conduct one or more of the assay methods of the present invention. Optionally, such kit may include one or more sets of instructions for use of the enclosed reagents, such as, for example, in a laboratory or clinical application.

“Link” or “join” refers to any method known in the art for functionally connecting one or more proteins, peptides, nucleic acids, or polynucleotides, including, without limitation, recombinant fusion, covalent bonding, disulfide bonding, ionic bonding, hydrogen bonding, electrostatic bonding, and the like.

As used herein, “matrix” shall mean a three-dimensional structure fabricated with biomaterials. The biomaterials can be biologically-derived or synthetic.

As used herein, a “medical prosthetic device,” “medical implant,” “implant,” and such like, relate to a device intended to be implanted into the body of a vertebrate animal, such as a mammal, and in particular a human. Implants in the present context may be used to replace anatomy and/or restore any function of the body. Examples of such devices include, but are not limited to, dental implants and orthopedic implants. In the present context, orthopedic implants include within its scope any devices intended to be implanted into the body of a vertebrate animal, in particular a mammal such as a human, for preservation and restoration of the function of the musculoskeletal system, particularly joints and bones, including the alleviation of pain in these structures.

In the present context, dental implants include any device intended to be implanted into the oral cavity of a vertebrate animal, in particular a mammal such as a human, in tooth restoration procedures. Generally, a dental implant is composed of one or several implant parts. For instance, a dental implant usually comprises a dental fixture coupled to secondary implant parts, such as an abutment and/or a dental restoration such as a crown, bridge, or denture. However, any device, such as a dental fixture, intended for implantation may alone be referred to as an implant even if other parts are to be connected thereto. Orthopedic and dental implants may also be denoted as orthopedic and dental prosthetic devices as is clear from the above. The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally-occurring animals.

As used herein, “mesh” means a network of material. The mesh may be woven synthetic fibers, non-woven synthetic fibers, nanofibers, or any combination thereof, or any material suitable for implantation into a mammal, and in particular, for implantation into a human.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally-occurring animals.

As used herein, the term “nucleic acid” includes one or more types of: polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including abasic sites). The term “nucleic acid,” as used herein, also includes polymers of ribonucleosides or deoxyribonucleosides that are covalently bonded, typically by phosphodiester linkages between subunits, but in some cases by phosphorothioates, methylphosphonates, and the like. “Nucleic acids” include single- and double-stranded DNA, as well as single- and double-stranded RNA. Exemplary nucleic acids include, without limitation, gDNA; hnRNA; mRNA; rRNA, tRNA, micro RNA (miRNA), small interfering RNA (siRNA), small nucleolar RNA (snORNA), small nuclear RNA (snRNA), and small temporal RNA (stRNA), and the like, and any combination thereof.

The term “operably linked,” as used herein, refers to that the nucleic acid sequences being linked are typically contiguous, or substantially contiguous, and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

As used herein, “osteoblast” shall mean a bone-forming cell which forms an osseous matrix in which it becomes enclosed as an osteocyte. It may be derived from mesenchymal osteoprogenitor cells. The term may also be used broadly to encompass osteoblast-like, and related, cells, such as osteocytes and osteoclasts. An “osteoblast-like cell” means a cell that shares certain characteristics with an osteoblast (such as expression of certain proteins unique to bones), but is not an osteoblast. “Osteoblast-like cells” include preosteoblasts and osteoprogenitor cells. Preferably the cells are from a compatible human donor. More preferably, the cells are from the patient (i.e., autologous cells).

As used herein, “osteointegrative” means having the ability to chemically bond to bone.

As used herein, the term “patient” (also interchangeably referred to as “host” or “subject”), refers to any host that can serve as a recipient of one or more of the therapeutic or diagnostic formulations as discussed herein. In certain aspects, the patient is a vertebrate animal, which is intended to denote any animal species (and preferably, a mammalian species such as a human being). In certain embodiments, a patient may be any animal host, including but not limited to, human and non-human primates, avians, reptiles, amphibians, bovines, canines, caprines, cavines, corvines, epines, equines, felines, hircines, lapines, leporines, lupines, murines, ovines, porcines, racines, vulpines, and the like, including, without limitation, domesticated livestock, herding or migratory animals or birds, exotics or zoological specimens, as well as companion animals, pets, or any animal under the care of a veterinary or animal medical care practitioner.

The phrase “pharmaceutically-acceptable” refers to molecular entities and compositions that preferably do not produce an allergic or similar untoward reaction when administered to a mammal, and, in particular when administered to a human. As used herein, “pharmaceutically acceptable salt” refers to a salt that preferably retains the desired biological activity of the parent compound and does not impart any undesired toxicological effects. Examples of such salts include, without limitation, acid addition salts formed with inorganic acids (e.g., hydrochloric acid, hydrobromic acid, sulfuric acid, phosphoric acid, nitric acid, and the like); and salts formed with organic acids including, without limitation, acetic acid, oxalic acid, tartaric acid, succinic acid, maleic acid, fumaric acid, gluconic acid, citric acid, malic acid, ascorbic acid, benzoic acid, tannic acid, pamoic (embonic) acid, alginic acid, naphthoic acid, polyglutamic acid, naphthalenesulfonic acids, naphthalenedisulfonic acids, polygalacturonic acid; salts with polyvalent metal cations such as zinc, calcium, bismuth, barium, magnesium, aluminum, copper, cobalt, nickel, cadmium, and the like; salts formed with an organic cation formed from N,N′-dibenzylethylenediamine or ethylenediamine; and combinations thereof.

The term “pharmaceutically-acceptable salt” as used herein refers to a compound of the present disclosure derived from pharmaceutically acceptable bases, inorganic or organic acids. Examples of suitable acids include, but are not limited to, hydrochloric, hydrobromic, sulfuric, nitric, perchloric, fumaric, maleic, phosphoric, glycollic, lactic, salicyclic, succinic, toluene-p-sulfonic, tartaric, acetic, citric, methanesulfonic, formic, benzoic, malonic, naphthalene-2-sulfonic, trifluoroacetic and benzenesulfonic acids. Salts derived from appropriate bases include, but are not limited to, alkali such as sodium and ammonia.

As used herein, the term “plasmid” or “vector” refers to a genetic construct that is composed of genetic material (i.e., nucleic acids). Typically, a plasmid or a vector contains an origin of replication that is functional in bacterial host cells, e.g., Escherichia coli, and selectable markers for detecting bacterial host cells including the plasmid. Plasmids and vectors of the present invention may include one or more genetic elements as described herein arranged such that an inserted coding sequence can be transcribed and translated in a suitable expression cells. In addition, the plasmid or vector may include one or more nucleic acid segments, genes, promoters, enhancers, activators, multiple cloning regions, or any combination thereof, including segments that are obtained from or derived from one or more natural and/or artificial sources.

As used herein, “polymer” means a chemical compound or mixture of compounds formed by polymerization and including repeating structural units. Polymers may be constructed in multiple forms and compositions or combinations of compositions.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures.

For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues described herein are preferred to be in the “L” isomeric form. However, residues in the “D” isomeric form may be substituted for any L-amino acid residue provided the desired properties of the polypeptide are retained.

As used herein, the terms “prevent,” “preventing,” “prevention,” “suppress,” “suppressing,” and “suppression” as used herein refer to administering a compound either alone or as contained in a pharmaceutical composition prior to the onset of clinical symptoms of a disease state so as to prevent any symptom, aspect or characteristic of the disease state. Such preventing and suppressing need not be absolute to be deemed medically useful.

As used herein, “porosity” means the ratio of the volume of interstices of a material to a volume of a mass of the material.

“Protein” is used herein interchangeably with “peptide” and “polypeptide,” and includes both peptides and polypeptides produced synthetically, recombinantly, or in vitro and peptides and polypeptides expressed in vivo after nucleic acid sequences are administered into a host animal or human subject. The term “polypeptide” is preferably intended to refer to any amino acid chain length, including those of short peptides from about two to about 20 amino acid residues in length, oligopeptides from about 10 to about 100 amino acid residues in length, and longer polypeptides including from about 100 amino acid residues or more in length. Furthermore, the term is also intended to include enzymes, i.e., functional biomolecules including at least one amino acid polymer. Polypeptides and proteins of the present invention also include polypeptides and proteins that are or have been post-translationally modified, and include any sugar or other derivative(s) or conjugate(s) added to the backbone amino acid chain.

“Purified,” as used herein, means separated from many other compounds or entities. A compound or entity may be partially purified, substantially purified, or pure. A compound or entity is considered pure when it is removed from substantially all other compounds or entities, i.e., is preferably at least about 90%, more preferably at least about 91%, 92%, 93%, 94%, 95%, 96%, 97%, 98%, 99%, or greater than 99% pure. A partially or substantially purified compound or entity may be removed from at least 50%, at least 60%, at least 70%, or at least 80% of the material with which it is naturally found, e.g., cellular material such as cellular proteins and/or nucleic acids.

The term “recombinant” indicates that the material (e.g., a polynucleotide or a polypeptide) has been artificially or synthetically (non-naturally) altered by human intervention. The alteration can be performed on the material within or removed from, its natural environment, or native state. Specifically, e.g., a promoter sequence is “recombinant” when it is produced by the expression of a nucleic acid segment engineered by the hand of man. For example, a “recombinant nucleic acid” is one that is made by recombining nucleic acids, e.g., during cloning, DNA shuffling or other procedures, or by chemical or other mutagenesis; a “recombinant polypeptide” or “recombinant protein” is a polypeptide or protein which is produced by expression of a recombinant nucleic acid; and a “recombinant virus,” e.g., a recombinant AAV virus, is produced by the expression of a recombinant nucleic acid.

The term “regulatory element,” as used herein, refers to a region or regions of a nucleic acid sequence that regulates transcription. Exemplary regulatory elements include, but are not limited to, enhancers, post-transcriptional elements, transcriptional control sequences, and such like.

The term “RNA segment” refers to an RNA molecule that has been isolated free of total cellular RNA of a particular species. Therefore, RNA segments can refer to one or more RNA segments (either of native or synthetic origin) that have been isolated away from, or purified free from, other RNAs. Included within the term “RNA segment,” are RNA segments and smaller fragments of such segments.

The term “a sequence essentially as set forth in SEQ ID NO:X” means that the sequence substantially corresponds to a portion of a sequence denoted, SEQ ID NO:X, and has relatively few nucleotides (or amino acids in the case of polypeptide sequences) that are not identical to, or a biologically functional equivalent of, the nucleotides (or amino acids) of SEQ ID NO:X. The term “biologically functional equivalent” is well understood in the art, and is further defined in detail herein. Accordingly, sequences that have about 85% to about 90%; or more preferably, about 91% to about 95%; or even more preferably, about 96% to about 99%; of nucleotides that are identical or functionally equivalent to one or more of the nucleotide sequences provided herein are particularly contemplated to be useful in the practice of the invention.

Suitable standard hybridization conditions for nucleic acids for use in the present invention include, for example, hybridization in 50% formamide, 5×Denhardt's solution, 5×SSC, 25 mM sodium phosphate, 0.1% SDS and 100 μg/mL of denatured salmon sperm DNA at 42° C. for 16 hr followed by 1 hr sequential washes with 0.1×SSC, 0.1% SDS solution at 60° C. to remove the desired amount of background signal. Lower stringency hybridization conditions for the present invention include, for example, hybridization in 35% formamide, 5×Denhardt's solution, 5×SSC, 25 mM sodium phosphate, 0.1% SDS and 100 μg/mL denatured salmon sperm DNA or E. coli DNA at 42° C. for 16 hr followed by sequential washes with 0.8×SSC, 0.1% SDS at 55° C. Those of ordinary skill in the art will recognize that such hybridization conditions can be readily adjusted to obtain the desired level of stringency for a particular application.

As used herein, “scaffold,” relates to an open porous structure. A scaffold may comprise one or more building materials to create the structure of the scaffold. Additionally, the scaffold may further comprise other substances, such as one or more biologically active molecules or such like.

As used herein, “soft tissue” is intended to include tissues that connect, support, or surround other structures and organs of the body, not being bone. Soft tissue includes ligaments, tendons, fascia, skin, fibrous tissues, fat, synovial membranes, epithelium, muscles, nerves and blood vessels.

As used herein, “stem cell” means an unspecialized cell that has the potential to develop into many different cell types in the body, such as mesenchymal osteoprogenitor cells, osteoblasts, osteocytes, osteoclasts, chondrocytes, and chondrocyte progenitor cells. Preferably, the cells are from a compatible human donor. More preferably, the cells are from the patient (i.e., autologous cells).

As used herein, the term “structural gene” is intended to generally describe a polynucleotide, such as a gene, that is expressed to produce an encoded peptide, polypeptide, protein, ribozyme, catalytic RNA molecule, or antisense molecule.

The term “subject,” as used herein, describes an organism, including mammals such as primates, to which treatment with the compositions according to the present invention can be provided. Mammalian species that can benefit from the disclosed methods of treatment include, but are not limited to, apes; chimpanzees; orangutans; humans; monkeys; domesticated animals such as dogs and cats; livestock such as horses, cattle, pigs, sheep, goats, and chickens; and other animals such as mice, rats, guinea pigs, and hamsters.

The term “substantially complementary,” when used to define either amino acid or nucleic acid sequences, means that a particular subject sequence, for example, an oligonucleotide sequence, is substantially complementary to all or a portion of the selected sequence, and thus will specifically bind to a portion of an mRNA encoding the selected sequence. As such, typically the sequences will be highly complementary to the mRNA “target” sequence, and will have no more than about 1, about 2, about 3, about 4, about 5, about 6, about 7, about 8, about 9, or about 10 or so base mismatches throughout the complementary portion of the sequence. In many instances, it may be desirable for the sequences to be exact matches, i.e., be completely complementary to the sequence to which the oligonucleotide specifically binds, and therefore have zero mismatches along the complementary stretch. As such, highly complementary sequences will typically bind quite specifically to the target sequence region of the mRNA and will therefore be highly efficient in reducing, and/or even inhibiting the translation of the target mRNA sequence into polypeptide product.

Substantially complementary nucleic acid sequences will be greater than about 80 percent complementary (or “% exact-match”) to a corresponding nucleic acid target sequence to which the nucleic acid specifically binds, and will, more preferably be greater than about 85 percent complementary to the corresponding target sequence to which the nucleic acid specifically binds. In certain aspects, as described above, it will be desirable to have even more substantially complementary nucleic acid sequences for use in the practice of the invention, and in such instances, the nucleic acid sequences will be greater than about 90 percent complementary to the corresponding target sequence to which the nucleic acid specifically binds, and may in certain embodiments be greater than about 95 percent complementary to the corresponding target sequence to which the nucleic acid specifically binds, and even up to and including about 96%, about 97%, about 98%, about 99%, and even about 100% exact match complementary to all or a portion of the target sequence to which the designed nucleic acid specifically binds.

Percent similarity or percent complementary of any of the disclosed nucleic acid sequences may be determined, for example, by comparing sequence information using the GAP computer program, version 6.0, available from the University of Wisconsin Genetics Computer Group (UWGCG). The GAP program utilizes the alignment method of Needleman and Wunsch (1970). Briefly, the GAP program defines similarity as the number of aligned symbols (i.e., nucleotides or amino acids) that are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess (1986), (2) a penalty of 3.0 for each gap and an additional 0.10 penalty for each symbol in each gap; and (3) no penalty for end gaps.

As used herein, the term “substantially free” or “essentially free” in connection with the amount of a component preferably refers to a composition that contains less than about 10 weight percent, preferably less than about 5 weight percent, and more preferably less than about 1 weight percent of a compound. In preferred embodiments, these terms refer to less than about 0.5 weight percent, less than about 0.1 weight percent, or less than about 0.01 weight percent.

As used herein, the term “substantially free” or “essentially free” in connection with the amount of a component preferably refers to a composition that contains less than about 10 weight percent, preferably less than about 5 weight percent, and more preferably less than about 1 weight percent of a compound. In preferred embodiments, these terms refer to less than about 0.5 weight percent, less than about 0.1 weight percent, or less than about 0.01 weight percent.

The terms “substantially corresponds to,” “substantially homologous,” or “substantial identity,” as used herein, denote characteristics of a nucleic acid or an amino acid sequence, wherein a selected nucleic acid sequence or a selected amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably, at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared.

As used herein, “synthetic” shall mean that the material is not of a human or animal origin.

The term “therapeutically-practical period” means the period of time that is necessary for one or more active agents to be therapeutically effective. The term “therapeutically-effective” refers to reduction in severity and/or frequency of one or more symptoms, elimination of one or more symptoms and/or underlying cause, prevention of the occurrence of symptoms and/or their underlying cause, and the improvement or a remediation of damage.

A “therapeutic agent” may be any physiologically or pharmacologically active substance that may produce a desired biological effect in a targeted site in a subject. The therapeutic agent may be a chemotherapeutic agent, an immunosuppressive agent, a cytokine, a cytotoxic agent, a nucleolytic compound, a radioactive isotope, a receptor, and a pro-drug activating enzyme, which may be naturally occurring, produced by synthetic or recombinant methods, or a combination thereof. Drugs that are affected by classical multidrug resistance, such as vinca alkaloids (e.g., vinblastine and vincristine), the anthracyclines (e.g., doxorubicin and daunorubicin), RNA transcription inhibitors (e.g., actinomycin-D) and microtubule stabilizing drugs (e.g., paclitaxel) may have particular utility as the therapeutic agent. Cytokines may be also used as the therapeutic agent. Examples of such cytokines are lymphokines, monokines, and traditional polypeptide hormones. A cancer chemotherapy agent may be a preferred therapeutic agent. For a more detailed description of anticancer agents and other therapeutic agents, those skilled in the art are referred to any number of instructive manuals including, but not limited to, the Physician's Desk Reference and Hardman and Limbird (2001).

As used herein, a “transcription factor recognition site” and a “transcription factor binding site” refer to a polynucleotide sequence(s) or sequence motif(s), which are identified as being sites for the sequence-specific interaction of one or more transcription factors, frequently taking the form of direct protein-DNA binding. Typically, transcription factor binding sites can be identified by DNA footprinting, gel mobility shift assays, and the like, and/or can be predicted based on known consensus sequence motifs, or by other methods known to those of ordinary skill in the art.

“Transcriptional regulatory element” refers to a polynucleotide sequence that activates transcription alone or in combination with one or more other nucleic acid sequences. A transcriptional regulatory element can, for example, comprise one or more promoters, one or more response elements, one or more negative regulatory elements, and/or one or more enhancers.

“Transcriptional unit” refers to a polynucleotide sequence that comprises at least a first structural gene operably linked to at least a first cis-acting promoter sequence and optionally linked operably to one or more other cis-acting nucleic acid sequences necessary for efficient transcription of the structural gene sequences, and at least a first distal regulatory element as may be required for the appropriate tissue-specific and developmental transcription of the structural gene sequence operably positioned under the control of the promoter and/or enhancer elements, as well as any additional cis-sequences that are necessary for efficient transcription and translation (e.g., polyadenylation site(s), mRNA stability controlling sequence(s), etc.

As used herein, the term “transformation” is intended to generally describe a process of introducing an exogenous polynucleotide sequence (e.g., a viral vector, a plasmid, or a recombinant DNA or RNA molecule) into a host cell or protoplast in which the exogenous polynucleotide is incorporated into at least a first chromosome or is capable of autonomous replication within the transformed host cell. Transfection, electroporation, and “naked” nucleic acid uptake all represent examples of techniques used to transform a host cell with one or more polynucleotides.

As used herein, the term “transformed cell” is intended to mean a host cell whose nucleic acid complement has been altered by the introduction of one or more exogenous polynucleotides into that cell.

“Treating” or “treatment of” as used herein, refers to providing any type of medical or surgical management to a subject. Treating can include, but is not limited to, administering a composition comprising a therapeutic agent to a subject. “Treating” includes any administration or application of a compound or composition of the invention to a subject for purposes such as curing, reversing, alleviating, reducing the severity of, inhibiting the progression of, or reducing the likelihood of a disease, disorder, or condition or one or more symptoms or manifestations of a disease, disorder, or condition. In certain aspects, the compositions of the present invention may also be administered prophylactically, i.e., before development of any symptom or manifestation of the condition, where such prophylaxis is warranted. Typically, in such cases, the subject will be one that has been diagnosed for being “at risk” of developing such a disease or disorder, either as a result of familial history, medical record, or the completion of one or more diagnostic or prognostic tests indicative of a propensity for subsequently developing such a disease or disorder.

The term “vector,” as used herein, refers to a nucleic acid molecule (typically comprised of DNA) capable of replication in a host cell and/or to which another nucleic acid segment can be operatively linked so as to bring about replication of the attached segment. A plasmid, cosmid, or a virus is an exemplary vector.

In certain embodiments, it will be advantageous to employ one or more nucleic acid segments of the present invention in combination with an appropriate detectable marker (i.e., a “label,”), such as in the case of employing labeled polynucleotide probes in determining the presence of a given target sequence in a hybridization assay. A wide variety of appropriate indicator compounds and compositions are known in the art for labeling oligonucleotide probes, including, without limitation, fluorescent, radioactive, enzymatic or other ligands, such as avidin/biotin, etc., which are capable of being detected in a suitable assay. In particular embodiments, one may also employ one or more fluorescent labels or an enzyme tag such as urease, alkaline phosphatase or peroxidase, instead of radioactive or other environmentally less-desirable reagents. In the case of enzyme tags, colorimetric, chromogenic, or fluorogenic indicator substrates are known that can be employed to provide a method for detecting the sample that is visible to the human eye, or by analytical methods such as scintigraphy, fluorimetry, spectrophotometry, and the like, to identify specific hybridization with samples containing one or more complementary or substantially complementary nucleic acid sequences. In the case of so-called “multiplexing” assays, where two or more labeled probes are detected either simultaneously or sequentially, it may be desirable to label a first oligonucleotide probe with a first label having a first detection property or parameter (for example, an emission and/or excitation spectral maximum), which also labeled a second oligonucleotide probe with a second label having a second detection property or parameter that is different (i.e., discreet or discernible from the first label. The use of multiplexing assays, particularly in the context of genetic amplification/detection protocols are well-known to those of ordinary skill in the molecular genetic arts.

Biological Functional Equivalents

Modification and changes may be made in the structure of the nucleic acids, or to the vectors comprising them, as well as to mRNAs, polypeptides, or therapeutic agents encoded by them and still obtain functional systems that contain one or more therapeutic agents with desirable characteristics. As mentioned above, it is often desirable to introduce one or more mutations into a specific polynucleotide sequence. In certain circumstances, the resulting encoded polypeptide sequence is altered by this mutation, or in other cases, the sequence of the polypeptide is unchanged by one or more mutations in the encoding polynucleotide.

When it is desirable to alter the amino acid sequence of a polypeptide to create an equivalent, or even an improved, second-generation molecule, the amino acid changes may be achieved by changing one or more of the codons of the encoding DNA sequence, according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the peptide sequences of the disclosed compositions or corresponding DNA sequences which encode said peptides without appreciable loss of their biological utility or activity.

TABLE 1

| | AMINO | | | | | | |
|---|---|---|---|---|---|---|---|
| | ACIDS | CODONS | | | | | |
| Alanine | Ala | GCA | GCC | GCG | GCU | | |
| Cysteine | Cys | UGC | UGU | | | | |
| Aspartic acid | Asp | GAC | GAU | | | | |
| Glutamic acid | Glu | GAA | GAG | | | | |
| Phenylalanine | Phe | UUC | UUU | | | | |
| Glycine | Gly | GGA | GGC | GGG | GGU | | |
| Histidine | His | CAC | CAU | | | | |
| Isoleucine | Ile | AUA | AUC | AUU | | | |
| Lysine | Lys | AAA | AAG | | | | |
| Leucine | Leu | UUA | UUG | CUA | CUC | CUG | CUU |
| Methionine | Met | AUG | | | | | |
| Asparagine | Asn | AAC | AAU | | | | |
| Proline | Pro | CCA | CCC | CCG | CCU | | |
| Glutamine | Gln | CAA | CAG | | | | |
| Arginine | Arg | AGA | AGG | CGA | CGC | CGG | CGU |
| Serine | Ser | AGC | AGU | UCA | UCC | UCG | UCU |
| Threonine | Thr | ACA | ACC | ACG | ACU | | |
| Valine | Val | GUA | GUC | GUG | GUU | | |
| Tryptophan | Trp | UGG | | | | | |
| Tyrosine | Tyr | UAC | UAU | | | | |

In making such changes, the hydropathic index of amino acids may be considered. The importance of the hydropathic amino acid index in conferring interactive biologic function on a protein is generally understood in the art (Kyte and Doolittle, 1982, incorporate herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index based on its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively based on hydrophilicity. U.S. Pat. No. 4,554,101 (specifically incorporated herein in its entirety by express reference thereto), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take one or more of the foregoing characteristics into consideration are well known to those of ordinary skill in the art, and include arginine and lysine; glutamate and aspartate; serine and threonine; glutamine and asparagine; and valine, leucine and isoleucine.

The section headings used throughout are for organizational purposes only and are not to be construed as limiting the subject matter described. All documents, or portions of documents, cited in this application (including, but not limited to, patents, patent applications, articles, books, and treatises) are expressly incorporated herein in their entirety by express reference thereto. In the event that one or more of the incorporated literature and similar materials defines a term in a manner that contradicts the definition of that term in this application, this application controls.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Microfluidic-Formulated Leukosomes Efficiently Deliver siRNA to Inflamed Endothelium

Recently, advances in biomimicry, i.e., the biologically inspired design of materials,^{[1]} has spurred the development of novel strategies to bestow nano- and micro-particles with multiple-functionalities necessary to negotiate biological barriers.^{[2]} Current approaches for drug delivery carriers include mimicking of leukocytes,^{[3]} red blood cells,^{[4]} platelets,^{[5]} and cancer cells^{[6]} to achieve superior delivery of therapeutics compared to conventional nanoparticles. These hybrid biomimetic carriers showed advantageous pharmaceutical properties (i.e., defined size and shape, physical stability, ability to load and release chemically-different therapeutics) resulting from the synthetic backbone materials (nano-porous silicon,^{[3a]} phospholipids,^{[3b]} PLGA^{[7]}) they were derived from. Furthermore, these biomimetic strategies demonstrated innate biological features and intrinsic functionalities (long circulation, selective targeting towards specific biological compartments) typical of the donor cell source.^{[8]} When investigating the events occurring at the nano-bio interface, e.g., the protein corona formation,^{[9]} a distinctive interaction of these biomimetic carriers with blood components compared to their non-biomimetic counterpart can be observed.^{[10]} Using this approach, leukocyte-like nanovesicles showed prolonged circulation and preferential targeting of inflamed vasculature,^{[3b]} while platelet-like nanoparticles displayed platelet-mimicking properties such as adhesion to damaged vasculature and binding to platelet-adhering pathogens.^{[5]} However, the new biological functionalities transferred to these hybrid nanomaterials increased their degree of complexity from a regulatory standpoint. Nanomaterials for biomedical applications must account for or develop methods to ensure that final products are standardized, batch-to-batch consistent, scalable, GMP-compliant, and amendable to high-throughput assembly methods. As a matter of fact, the difficulty of producing nanoparticles in a standardized and reproducible way in sufficient quantities has hindered their successful translation to clinical applications. However, despite current protocols addressing several hurdles, such as: i) retention of biological complexity of cellular membrane on carrier surface, ii) control of physicochemical properties over the final formulation, iii) customizability and iv) stability, a major challenge remains in the development of adequate protocols for scaling up the manufacturing of nanoparticles.^{[11]}

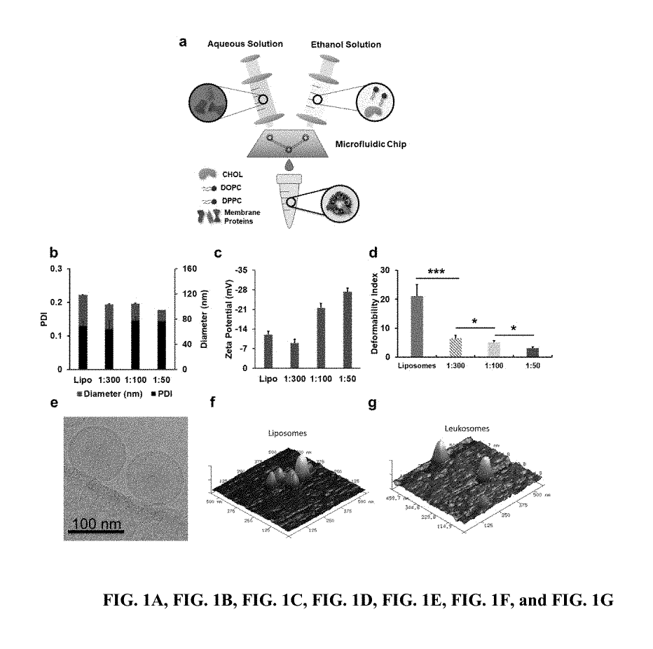

In response to this need, microfluidics—the science of manipulating fluids at the micrometer or smaller scale in a controlled fashion^{[12]}-emerged as a promising technique allowing for the controlled synthesis of nanoparticles providing a versatile method to accelerate their transition to the clinic.^{[11]} From a technical standpoint, the concept behind microfluidics is that a change in solvent polarity can drive the self-assembly of lipids or other amphiphilic molecules.^{[13]} By controlling the flow rate ratio (FRR) between the aqueous and organic phases, the total flow rate (TFR) of the two streams, and the temperature, it is possible to tune the final size and distribution of resulting nanoparticles^{[11, 14]} as well as their drug loading capacity and batch-to-batch reproducibility.^{[13, 15]} Recently, a microfluidic-based platform, called NanoAssemblr™ (NA) (FIG. 1A), has been developed for the manufacture of nanoparticles in a controlled, tunable, low-cost, and scalable fashion.^{[16]} The mixing process in the microfluidic mixing chamber of the NA is achieved through the combination of a Y-shaped inlet channel and the inclusion of microstructures, so-called herringbone mixers.^{[17]} The herringbone micromixer induces chaotic advection, which allows for the stretching and folding of fluid streams over the channels' cross-sectional area. This, together with the herringbone structures of the channel floor, increases mass transfer under laminar flow conditions.^{[18]} The repeated folding of two miscible fluids under laminar flow allows for extremely fast mixing (millisecond mixing) of the two fluids under mild conditions (low shear, low heat, and low pressure) and prevents the occurrence of uncontrolled solvent gradients. The adjustment of mixing ratio, flow rate, and lipid composition allowed for the fine-tuning of physical features of lipid nanoparticles for the delivery of adjuvants and siRNA.^{[19]}

Herein, it is shown, for the first time, a continuous-based process to incorporate membrane proteins derived from leukocytes within the lipid bilayer of liposome-like nanovesicles (i.e., leukosomes) using NA technology. This study represents the first demonstration of the versatile, reproducible, robust and high-throughput manufacture of biomimetic nanovesicles using a microfluidic-based synthetic protocol. NA-formulated Leukosomes (NA-Leuko) produced by this method have been fully characterized for their physical and pharmaceutical properties. In addition, the successful transfer of biological features to NA-Leuko has been validated using both in vitro and in vivo models.^{[3b]}

Materials and Methods

### Materials

The NanoAssemblr™ platform (Precision NanoSystems, Inc., Vancouver, CANADA) was used for incorporating leukocyte membrane proteins in lipid nanovesicles (FIG. 1A). The lipids used in this study: 1,2-dipalmitoyl-sn-glycero-3-phosphocholine (DPPC), 1,2-dioleoyl-sn-glycero-3-phosphocholine (DOPC) and cholesterol were purchased from Avanti Polar Lipids, Inc., (Alabaster, Ala., USA) (purity >99%). HPLC-grade solvents were purchased from Fisher Scientific (Leicestershire, UNITED KINGDOM). Antibodies for flow cytometry (FITC-labeled anti-LFA-1, PerCP-labeled anti-CD45, COX IV, p62, and WGA) were obtained from BD Biosciences. CD3z antibody was purchased from Santa Cruz.

Membrane Protein Extraction and Incorporation within Lipid Bilayers Using Microfluidic Mixer

Biological studies aimed to proof NA-Leuko biological properties have been performed in syngeneic conditions. Leukocytes from human blood and immortalized J774 murine macrophages^{[3b]} have been used to assemble human and murine nanovesicles (NA-Leuko) for studies involving either human (in vitro) or murine (in vitro and in vivo) settings, respectively. While murine membrane proteins were obtained from the immortalized J774 macrophage cell line as previously shown,^{[3]} human membrane proteins were extracted from whole blood leukocytes, isolated following RBC lysis and centrifugation as reported from other investigators.^{[29]}

Phosphocholine-based phospholipids (DPPC and DOPC) and cholesterol (4:3:3 molar ratio) were dissolved in ethanol at a final lipid concentration of 9 mM and loaded in the organic phase inlet (FIG. 1A—Ethanol solution syringe). Membrane proteins, instead, were resuspended in aqueous buffer at 1:50, 1:100, or 1:300 protein-to-lipid concentrations and loaded in the second inlet (FIG. 1A—Aqueous solution syringe). Before proceeding with incorporation of membrane proteins, aqueous buffer and the ethanol solution of lipids were mixed at different flow rates (TFR) and flow ratios (FRR) between the two inlet streams to identify the conditions leading to the most consistent formulations in terms of size, size homogeneity, stability, and protein incorporation. Once prepared, formulations were purified from ethanol by either dialysis or ultracentrifugation methods. Control liposomes were assembled with the NanoAssemblr™ Benchtop platform using the following settings: 2:1 FRR, 1 mL/min TFR, and 45° C. Physical characterization of NA-Leuko and computational model analyses are discussed below. Experiments were performed on at least three different batches of independently-synthesized particles.

### In Vitro Adhesion of Human NA-Leuko to a Reconstructed Endothelium in Flow Condition

Adhesion experiments using HUVEC cells were carried out using human NA-Leuko. Rhodamine-labeled NA-Leuko and liposomes, resuspended in EBM-2 medium, were then infused into the slides containing HUVEC cells using a Harvard Apparatus PHD 2000 Infusion syringe pump at a speed of 100 μL min⁻¹ for 30 min. After infusion, cells were washed in PBS then fixed for 10 min using 4% paraformaldehyde at room temperature. The nuclei were stained for 1 min with a PBS solution containing 4′,6-diamidino-2-phenylindole (DAPI) and washed to remove any free DAPI. Cells were imaged using an inverted Nikon Eclipse Ti fluorescence microscope equipped with a Hamamatsu ORCA-Flash 2.8 digital camera.

### In Vivo Inflamed Endothelium Targeting of NA-Leuko

All animal experiments were performed in accordance with the guidelines of the Animal Welfare Act and the Guide for the Care and Use of Laboratory Animals approved by The Houston Methodist Institutional Animal Care and Use Committee guidelines (Houston, Tex., USA). Six to eight-week-old Balb/c mice (Charles River Laboratories, Wilmington, Mass., USA) were injected in the right ear with 10 μL of LPS (5 mg/mL solution, 50 μg/ear). 100 μL of rhodamine-labeled Leuko-NA were administrated 30 min after LPS injection. Mouse ears (healthy and inflamed) were prepared for intravital microscopy (IVM) imaging at 1 and 24 hr after injection to assess nanoparticles accumulation. Before IVM imaging, mice were injected with a 70 kDa FITC-dextran (50 μL in PBS, Invitrogen) to identify the vasculature in the ear. IVM was performed with an upright Nikon MR laser scanning confocal microscope equipped with a resonance scanner, motorized and heated stage, and Nikon long working distance 4× and 20× dry plan-apochromat objectives and is housed within the Intravital Microscopy Core at Houston Methodist Research Institute.

### Statistical Analysis

GraphPad statistical software (La Jolla, Calif., USA) was used to assess statistical significance between groups. Student's test and one-way ANOVA test were applied to compare differences between groups. A value of p=0.05 was considered statistically significant. For inflamed ear targeting, unpaired two-tailed t-tests assuming both population have equal SD were used to compare means of fold change in accumulation at 1 and 24 hr.

### Physical Characterization of NA-Leuko

Vesicle size and polydispersity index were determined through dynamic light scattering analysis using a Nanosizer ZS (Malvern Instruments). Surface charge (Zeta potential) was measured using a ZetaSizer Nano ZS (Malvern Instruments, Malvern, UNITED KINGDOM). Samples were diluted 1:50 in water for the analysis. Results are presented as average of at least 5 measurements, 10 runs each. For CryoEM analysis, NA-Leukosomes were plunge-frozen on holey film grids (R2×2 Quantifoil®; Micro Tools GmbH, Jena, GERMANY) as previously reported.^{[30]} Images were acquired on a JEOL 2100 electron microscope under low electron-dose conditions (˜5-20 electrons/Å²) using a 4,096×4,096 pixel CCD camera (UltraScan 895, GATAN, Inc., nominal magnifications 20,000×).

Evaluation of Protein Integration and Orientation into Leukosome's Lipid Bilayer

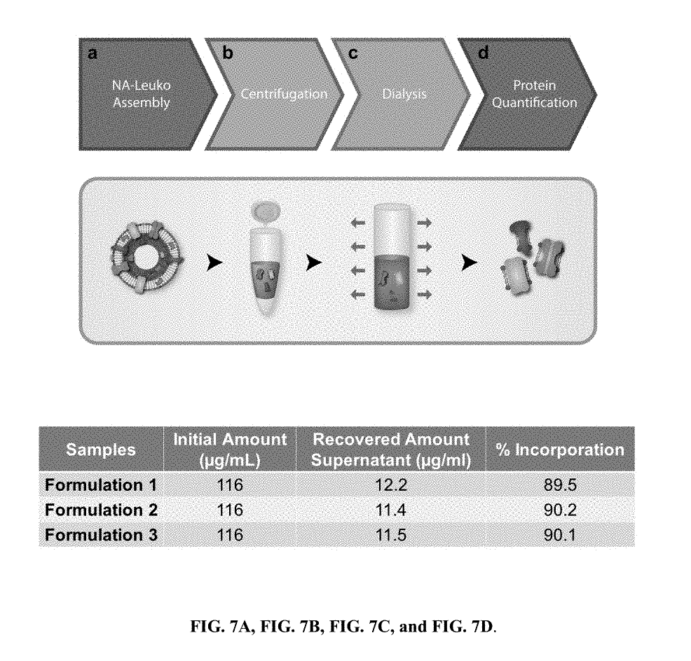

In this study, vesicle deformability following increasing protein-to-lipid concentrations, from 1:300 to 1:100 and 1:50, was measured using the extrusion assay.^{[30b]} The percentage of membrane proteins' integration within NA-Leuko was measured indirectly through the Bradford assay, as represented in the schematic in FIG. 7A, FIG. 7B, FIG. 7C, and FIG. 7D. Briefly, three independently-assembled batches of NA-Leuko were ultracentrifuged to isolate the unincorporated membrane proteins (dispersed into the supernatant). To eliminate the unincorporated lipids and avoid any interference with the Bradford reagent, the supernatant was dialyzed using Amicon filters. Once lipids were removed, the supernatant was analyzed with the Bradford assay. The percentage of incorporation has been calculated using the following equation:

$$\% of Incorporation = 100 - \frac{As}{Ai} \times 100 ;$$

where Ai is the amount (expressed in μg×mL⁻¹) of membrane proteins initially added to the formulation according to the 1:50 protein-to-lipid ratio and As is the amount (expressed in μg×mL⁻¹) of membrane proteins calculated using the Bradford assay.

The presence of leukocyte-derived membrane proteins on NA-Leuko and their correct orientation was validated using flow cytometry analysis.^{[30b]} CD45 and LFA-1 were selected as representative markers. NA-Leuko and control liposomes were diluted in FACS Buffer (PBS, 1% BSA) to a final concentration of 0.5 mM and incubated separately with FITC-labeled anti-LFA-1 and PerCP-labeled anti-CD45 (2.5 μg mL⁻¹) designed to bind the protein's extracellular domain for at least 30 min at room temperature. Samples were next dialyzed using 1000 kDa membrane filters for 1.5 hr in water covered from light with mild stirring and then analyzed on the flow cytometer. In addition, the presence of intracellular domains of membrane proteins, before and after bilayer permeabilization, was also evaluated using CD3z antibody through flow cytometry analysis as previously reported.^{[31]}

### Theoretical Calculations of NA-Leuko Surface Area and Membrane Proteins Density Compared to TLE-Leuko:

Density of lipids:

ρ=1.43 g/cm³

Gram of lipids for NA-Leuko:

g_{NA}=0.058 g

Gram of lipids for TLE-Leuko:

g_{TLE}=0.02 g

### Radius of NA-Leuko:

r=47 nm

### Radius of TLE-Leuko:

r=61 nm

### Mass per NA-Leuko:

$${M}_{NA} = ρ \times \frac{4}{3} π {r}_{( NA )}^{3} = 6.4 \times {10}^{- 16} g per nanovesicle$$

### Mass per TLE-Leuko:

$${M}_{TLE} = ρ \times \frac{4}{3} π {r}_{( TLE )}^{3} = 1.4 \times {10}^{- 15} g per nanovesicle$$

### Number of NA-Leuko:

N_{NA}=9×10¹²

### Number of TLE-Leuko:

N_{TLE}=1.4×10¹³

Number of NA-Leuko per g of lipids:

N_{NAg}=1.5×10¹⁵

Number of TLE-Leuko per g of lipids:

N_{TLEg}=7.1×10¹⁴.

Surface area of each NA-Leuko:

S_{NA}=4πr_{(NA)}²=0.027 μm² per nanovesicle.

Total surface area of NA-Leuko per g of lipids:

S_{NAtotal}=N_{NA}×S_{NA}=4.3×10¹³ μm².

Surface area of each TLE-Leuko:

S_{TLE}=4πr_{(TLE)}²=0.046 μm per nanovesicle.

Total surface area of TLE-Leuko per g of lipids:

S_{TLEtotal}=N_{TLE}×S_{TLE}=3.3×10¹³ μm².

Membrane proteins incorporated into NA-Leuko:

M_{NAi}=104.3 μg.

Membrane proteins incorporated into TLE-Leuko:

MP_{TLEI}=41.96 μg.

Membrane proteins incorporated into NA-Leuko per g of lipids:

$${MP}_{NAg} = \frac{{M}_{NAi}}{{N}_{NA} \times {g}_{NA}} = 1.99 \times {10}^{- 9} µg per g lipids$$

Membrane proteins incorporated into TLE-Leuko per g of lipids:

$${MP}_{TLEg} = \frac{{M}_{TLEi}}{{N}_{TLE} \times {g}_{TLE}} = 1.48 \times {10}^{- 10} µg per g lipids$$

Density of membrane proteins on NA-Leuko surface per g of lipids:

$${NA}_{d} = \frac{{M}_{NAi}}{{S}_{NAtotal} \times {g}_{NA}} = 7.19 \times {10}^{- 8} µg membrane proteins per {µm}^{2} surface area .$$

Density of membrane proteins on TLE-Leuko surface per g of lipids:

$${TLE}_{d} = \frac{{M}_{TLEi}}{{S}_{TLEtotal} \times {g}_{TLE}} = 3.16 \times {10}^{- 9} µg membrane proteins per {µm}^{2} surface area .$$

### Atomic Force Microscopy Analysis

AFM images of NA-Leuko were collected in Scan Asyst® mode by Multimode (Bruker, Calif., USA) using single-beam silicon cantilever probes (Bruker MLCT: resonance frequency 10 KHz, nominal tip radius of curvature 10 nm, force constant of 0.04 N/m) and compared to control liposomes. Particles' roughness (Ra), expressed as arbitrary units (a.u.), was calculated using Nanoscope 6.13R1 software (Digital Instruments, NY, USA). Mean values from 60 random particles in 3 independent experiments are reported. In addition, Young's elastic modulus was determined in order to determine viscoelastic properties of particles using the following equation as previously reported.^{[30b, 32]}

F−F_{adh}=4/3E*\sqrt{R(d−d₀)³}

### Detection of Contaminants

The handling of biological material poses serious risks of nanotoxicology issues due to the presence of intracellular contaminants. The presence of nuclear and mitochondrial impurities into NA-Leuko samples has been investigated. NA-Leuko formulation was analyzed for the presence of nuclear (p62 staining) and mitochondrial (COX IV) proteins, as well as for genetic material (DAPI staining), according to the above described method for evaluation of protein integration and orientation into leukosome's lipid bilayer.

### Evaluation of Protein Glycosylation on NA-Leuko Surface

The wheat germ agglutinin (WGA) assay (Life Technologies, San Diego, Calif., USA) was carried out to evaluate membrane proteins glycosylation. Control liposomes and NA-Leuko were incubated with Alexa Fluor® 488-conjugated WGA (1 μg/mL) in standard buffers (HBSS) for 10 min and then washed through dialysis. WGA fluorescence (excitation/emission maxima 495/519 nm) was measured through spectrofluorimetric analysis.

### Evaluation of Physical Stability by Turbiscanlab Expert™

The stability of vesicular systems has been investigated as a function of both the temperature and incubation time by means of Turbiscan Lab® (Formulaction, Toulouse, FRANCE). Turbiscan Lab® is an optical analyzer that is able to predict the potential destabilization of colloidal carriers using the multiple-light-scattering analysis.^{[33]} The instrument was equipped with a throbbing luminous source at a near-infrared wavelength (880 nm) and two synchronous detectors (transmittance and backscattering). The samples were filled into cylindrical glass cells and then analysed (2 mg of lipids/mL). The transmittance (T) is the detection of the photon (λ), which crosses the sample (at 0° to the incident radiation); while the backscattering (BS) is the detection of the photon (λ) scattered by the sample (at 135° to the incident radiation). The instrument was calibrated using a reflectance standard (polystyrene lattice, totally opalescent) and a transmittance standard (silicon oil, totally transparent). Results are the transmitted or reflexed photons (as a percentage) compared to standards. The analysis was carried out every minute for 1 hr as reference mode. Data were processed using TurbiSoft 2.0 software and reported as kinetic stability profile versus time.

### In Vitro Adhesion of Human NA-Leuko to a Reconstructed Endothelium in Flow Condition

Adhesion experiments using HUVEC cells have been carried out using human NA Leuko. Flow experiments were performed by seeding HUVEC cells onto ibidi μ-slide I^{0.4} Luer ibiTreat, tissue culture treated slides. Briefly, slides were incubated for 30 min with fibronectin at a concentration of 50 μg mL⁻¹. HUVEC were then seeded at 1.25×10⁶ cell mL⁻¹ and incubated for 24 hr. Slides were then washed by slowly passing PBS into the wells. Rhodamine-labeled NA-Leuko and liposomes, resuspended in EBM-2 medium, were then infused into the slides using a Harvard Apparatus PHD 2000 Infusion syringe pump at a speed of 100 μL min⁻¹ for 30 min. After infusion was complete, cells were briefly washed in PBS then fixed for 10 min using 4% paraformaldehyde at room temperature. Nucleii were then stained by infusing cells for 1 min with a PBS solution containing 4′,6-diamidino-2-phenylindole (DAPI), then washed to remove any free DAPI. Cells were then left in PBS and immediately images using an inverted Nikon Eclipse Ti fluorescence microscope equipped with a Hamamatsu ORCA-Flash 2.8 digital camera.

Computational Model of Membrane Protein Incorporation within Lipid Bilayer

Starting from the X-ray crystallographic coordinates of H-2DD MHC CLASS I, deposited in the Protein Data Bank (PDB) with the code 1BII^{[34]}, and after adding the missing residues, the glycosylation sites were identified at the amino acid positions 110 and 200 on the experimental protein with entry ID P01900 collected from UNIPROT database. The protein was incorporated between T287 and T310 residues of the lipid bilayer transmembrane (TM) portion.

A Martini Maker tool^{[35]} was implemented in CHARMM-GUI^{[36]}, to generate the computational system and input files for GROMACS molecular dynamics package, version 5.1.4. All simulations were performed using the Martini 2.2 force field.^{[37]} The building procedure of membrane systems in Martini Maker provided the general steps in CHARMM-GUI Membrane and Vesicle Builder. After uploading the PDB structure file containing the prepared structure, the protein was inserted into the vesicle system between Thr287 and Met310 residues. To evaluate the different orientations of the protein in the lipid bilayer, two molecular systems were generated with both the extracellular and intracellular domain directed towards the inside of the vesicle curvature, flipping the protein along the z-axis.

For this study, the vesicle system was set as follow: rectangular bow type; DBPC:1, DOPC:3, DPPC:5 and CHOL:1 ratio of lipid components; 40 and 90 Å, respectively, for the water thickness and the vesicle radius. The resulting system contained 1741 lipids in the upper leaflet and 1126 in the lower leaflet. The solvent comprised of 184146 Martini water molecules and 5 Na⁺ ions. The assembled structures were converted to coarse grained representation by using the martinize.py script.^{[38]} All the components assembled to form a starting point simulation system and the simulation input files were generated. In explicit-solvent systems from Vesicle Builder, the Lennard-Jones and Coulombic interactions were smoothly switched off at 0.9 and 1.2, respectively. The systems were energy-minimized (steepest descent, 3000 steps). The generated system was relaxed in 30 ns long simulation. After equilibration, production simulations were run for 0.5 μs. These were run using isotropic pressure coupling maintaining the system at 1 bar using the Berendsen barostat^{[39]} with a time constant τp=5.0 ps, and a compressibility of 4.5.10-5 bar for bilayer system. The temperature was maintained at 303.15 K using the velocity rescaling thermostat of Bussi et al.^{[40]} with a τT=1.0 ps. The relative dielectric constant was 15 for system with no-polarizable water.

Results and Discussion

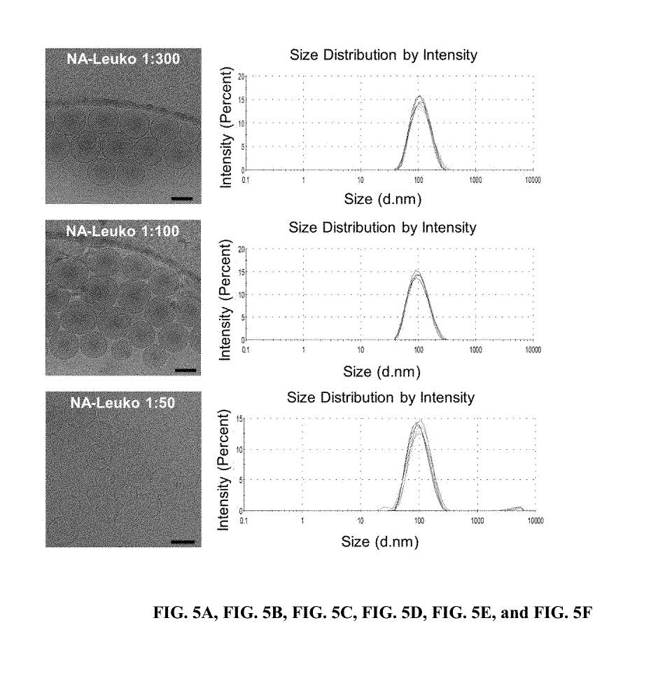

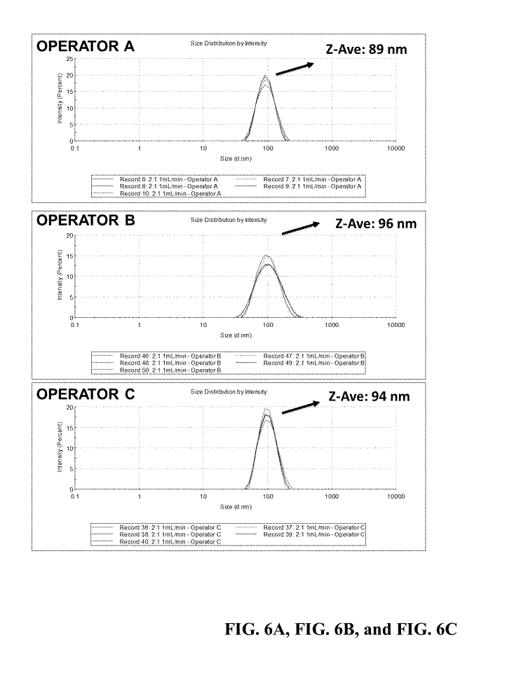

The first step in optimizing NA-Leuko assembly consisted of tailoring mixing protocols to generate stable nanovesicles suitable for additional membrane protein incorporation in terms of average diameter, size homogeneity, and zeta potential. By tuning FRR (1:1, 2:1, and 3:1 aqueous-to-organic phase), TFR (1, 3, 6, 9, and 12 mL min⁻¹), and operating temperature (room temperature versus 45° C.), a 2:1 FRR, 1 mL/min TFR, and a reaction temperature of 45° C. gave the best conditions for membrane protein incorporation. Liposomes produced with these settings showed a mean diameter of 118 nm, a polydispersity index (PDI) of 0.13, and surface charge of −12 mV (FIG. 1A-FIG. 1G). The incorporation of membrane proteins at increasing protein-to-lipid ratios (from 1:300, 1:100, to 1:50) induced a slight reduction of the mean diameter of the resulting proteolipid vesicles, i.e., from 118 nm (control liposomes) to 103, 104, and 94 nm, respectively (FIG. 1B), while significantly affecting their surface charge (FIG. 1C). Contrarily to observations using the thin layer evaporation (TLE) method,^{[3b]} protein integration into the lipid bilayer produced a relative decrease of surface charge (e.g., −9, −21, and −27 mV, respectively) that, in the case of NA-Leuko, was proportional to the increase in the protein-to-lipid concentration (FIG. 1C). A minor increase in PDI (<0.2 for all protein-to-lipid ratios) following protein incorporation, instead, was observed (FIG. 1B), revealing a high size homogeneity for these formulations. Size homogeneity were also confirmed by low magnification cryoEM analysis (see FIG. 5A, FIG. 5B, FIG. 5C, FIG. 5D, FIG. 5D, and FIG. 5E). It is worth noting that the above-mentioned values result from the average of at least three different batches of independently synthesized particles, which reveal the high reproducibility of this method, as previously reported by others.^{[20]} To further confirm the small batch-to-batch variations, the reproducibility of NA-Leuko assembled (i.e., size distribution) by three different operators was evaluated. As reported in FIG. 6A, FIG. 6B, and FIG. 6C, the maximum variability observed among three individual operators was 2.5%, indicating consistent reproducibility of NA-leuko.

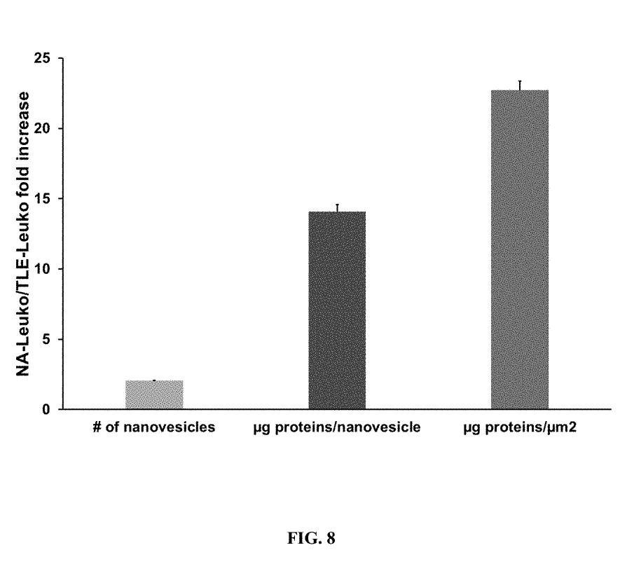

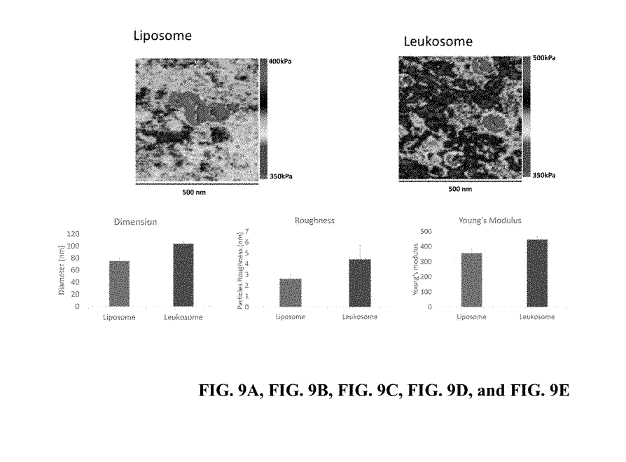

Next, the extrusion assay was performed to determine the successful incorporation of membrane proteins within the lipid bilayer. As described previously,^{[3b]} a direct correlation was observed between the bilayer transition temperature (Tₘ) and its physical deformability, following protein integration within a lipid bilayer. It was found that the increase of Tₘ, which is directly related to the increase of membrane protein incorporation, corresponded to a reduction of bilayer deformability. In other words, the higher the protein incorporation, the more rigid the lipid bilayer, and the less deformable the vesicles. As shown in FIG. 1D, a reduction of vesicle deformability, expressed as the deformability index (DI), was observed that was proportional to the increase of protein concentration in the following order: 1:50<1:100<1:300. While statistical significance was calculated upon increasing protein-to-lipid ratio from 1:300 to 1:50, the most optimal ratio was found to be 1:50, as a plateau was eventually reached at higher concentrations, indicating minimal gain from the increased protein composition. Taken together, these findings reveal that 1:50 protein-to-lipid ratio represents the highest level of protein incorporation and, per these results, 1:50 protein-to-lipid ratio was selected as the best ratio for further studies. The evaluation of the percentage of protein incorporation into the bilayer of three independently-assembled NA-Leuko revealed that, compared to the TLE method^{[3b]} (protein incorporation efficiency of 63%), around 90% of the membrane proteins initially added to the aqueous stream are associated to the final formulation (see FIG. 7A, FIG. 7B, FIG. 7C, and FIG. 7D). In addition, theoretical calculations based on established criteria performed on NA-Leuko vs. TLE-Leuko showed i) a 2.18-fold increase of total number of nanovesicles per gram of lipid; ii) a 14-fold increase of membrane proteins into the NA-Leuko bilayer with respect to TLE-Leuko; and iii) a 22.7-fold increase of membrane proteins per μm² of surface area for NA-Leuko compared to TLE-Leuko (see FIG. 8). Inspection of CryoEM images of NA-Leuko at this ratio revealed spherical shape and unilamellarity (FIG. 1E). Atomic force microscopy (AFM) analysis validated the size of the particles as measured with DLS and cryoEM (FIG. 1F and FIG. 1G). Additional AFM analysis showed increased roughness and elasticity for NA-Leuko compared to control liposomes (see FIG. 9A, FIG. 9B, FIG. 9C, FIG. 9D, and FIG. 9E), but similar to TLE Leukosomes (TLE-Leuko).^{[3b]}

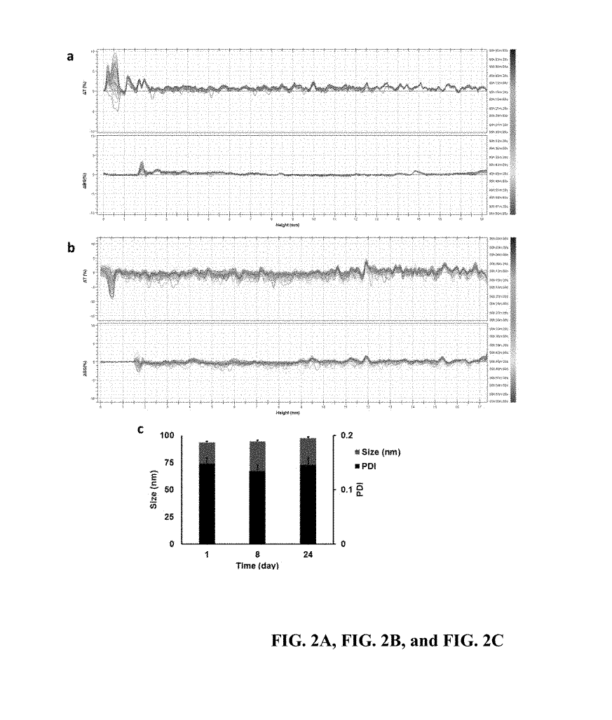

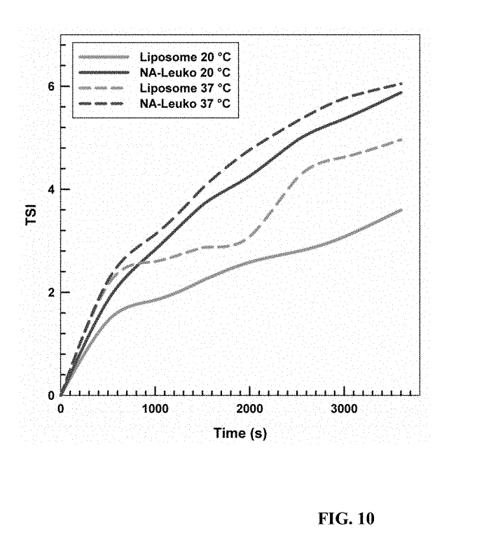

The physical stability of NA-Leuko was evaluated for pharmaceutical purposes through Turbiscan™ Lab.^{[21]} Widely-used in the pharmaceutical field, this method uses the multiple-light-scattering principle to detect any instability phenomena of colloidal systems (e.g., flocculation, sedimentation, and coagulation) through the analysis of photons scattered (delta backscattering, ABS) or transmitted (delta transmittance, AT) from the sample over time.^{[21-22]} The ABS and AT profiles were lower than ±5% for both control liposomes and NA-Leuko at 20° C., thus indicating a long-term shelf life^{[23]} (FIG. 2A and FIG. 2B). To support this finding, NA-Leukosomes were stored in solution at 4° C. up to 24 days and their size was measured over time. DLS analysis revealed minimal changes in diameter and size homogeneity (FIG. 2C). In addition, the evaluation of the turbiscan stability index (TSI) revealed similar profiles between the two formulations at both 20° C. and 37° C., thus confirming that proteins incorporation had no unfavorable effect on the stability of NA-Leuko (see FIG. 10).

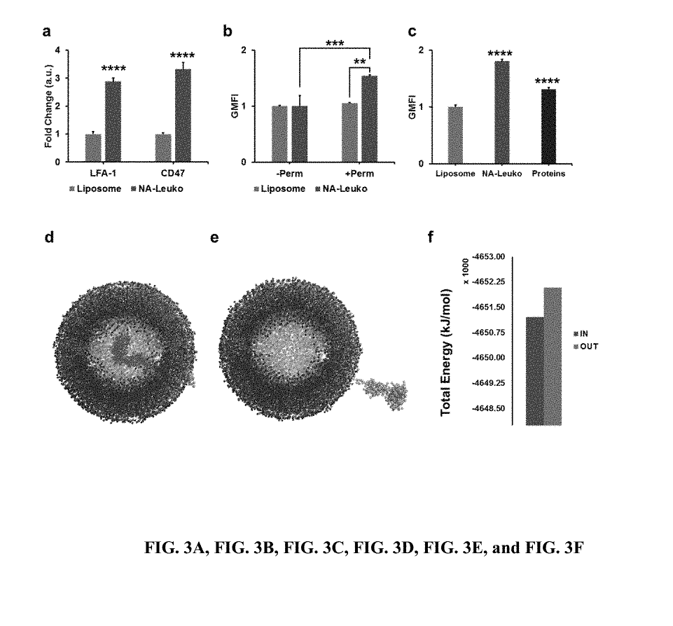

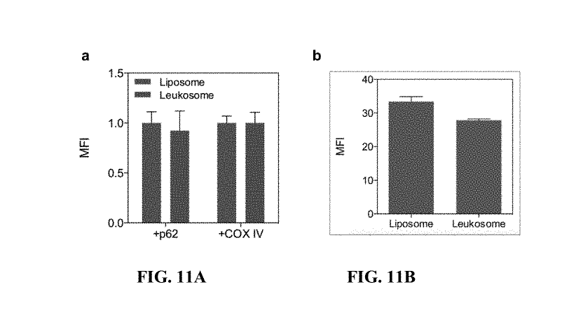

To evaluate the impact of microfluidic manipulation on the orientation of proteins within NA-Leuko bilayer. The exposure of the extracellular domains of LFA-1 and CD47 was confirmed on the surface of NA-Leuko (FIG. 3A). In addition, analysis of CD3z^{[3a]} revealed the absence of intracellular domains of membrane proteins exposed on the outer leaflet of NA-Leuko bilayer (FIG. 3B). As for control liposomes, no significant difference was observed in CD3z signal, while after bilayer permeabilization using 0.01% Tween 80, an increase in CD3z signal was observed only for NA-Leuko (FIG. 3B). Further assays were carried out to detect the presence of any contaminants in NA-Leuko formulation. Flow cytometry analysis did not detect p62 and COX IV markers, representative of nuclear and mitochondrial contaminants, respectively (see FIG. 11A). In addition, no statistically-significant difference between liposomes and NA-Leuko was observed after DAPI incubation, confirming the absence of contamination from nucleic acids (see FIG. 11B). Wheat germ agglutinin assay revealed the presence of glycosylated domains of membrane proteins (FIG. 3C), suggesting both the retention of post-translational modifications as well as the correct orientation of the surface proteins. While proteins incorporation within a lipid bilayer may at first appear random, it is believed that various factors, like glycosylation,^{[3b, 24]} the steric hindrance of the protein extracellular domain versus the intracellular domain and relative to vesicle curvature, are critical factors that drive their correct orientation.

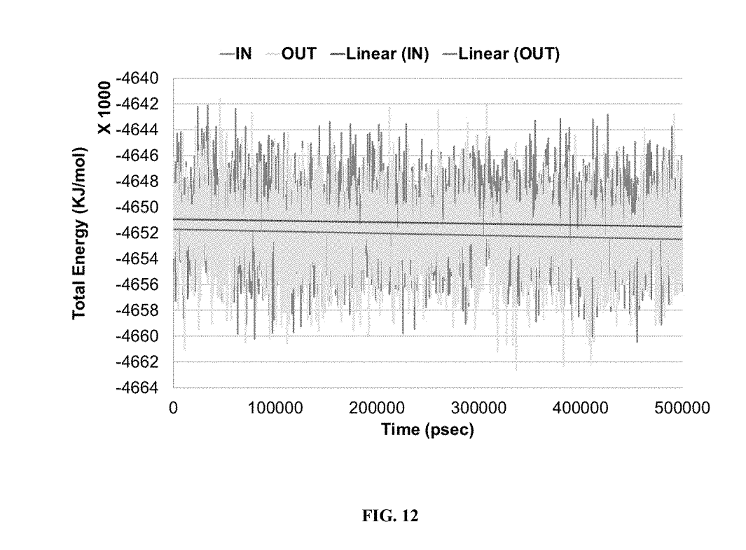

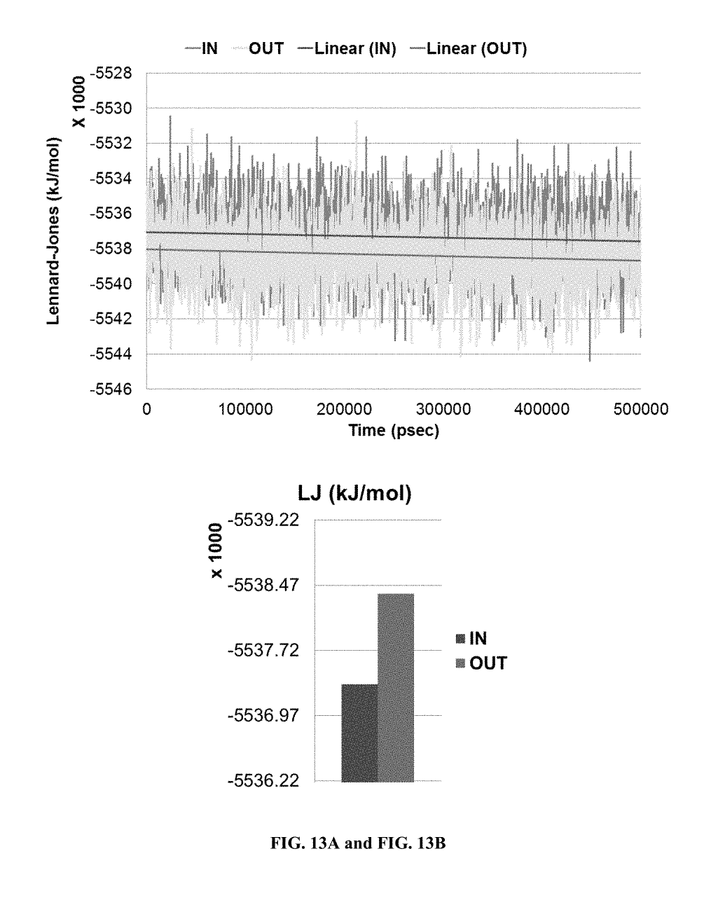

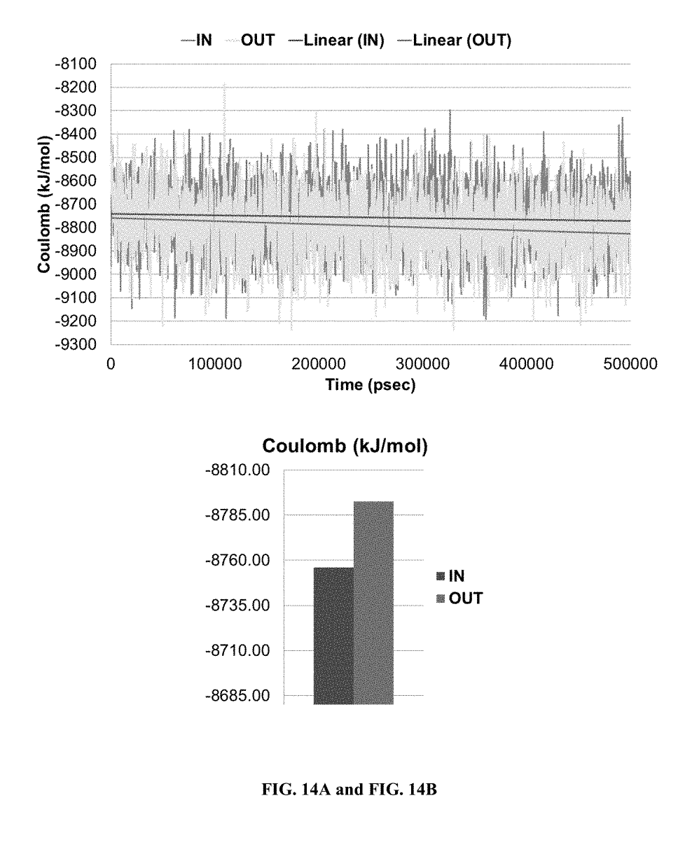

In silico analysis was performed using a simplified system that simulates the thermodynamic profile of an integral protein within the lipid bilayer of a leukosome. The computational analysis does not directly demonstrate the dynamics beyond the orientation of the membrane proteins into the NA-Leuko bilayer, but supports theoretically the experimental findings obtained through physical characterization and flow cytometry analysis. In this scenario, the use of coarse grained (CG) models has proven to be a valuable tool to probe the time and length scales of systems beyond what is feasible with the traditional computational models (e.g., all atomistic models)^{[25]}. CG simulations allowed for calculating free energy barriers with similar accuracy as those from the full atomistic ones, while accelerating 500-fold the simulation, and preserving the biological relevance of the interactions.^{[25]} To discriminate the energetic component that most likely stabilized the system in one of the two orientations, the potential scenario having the glycosylated domain exposed outside the bilayer (orientation OUT) or inside, towards the vesicle core (orientation IN) was simulated. The CG model (FIG. 3D and FIG. 3E) was very useful to evaluate the generated system not only for the low number of particles, but also for the low potential energy background, which allowed us to explore longer time lapses. The system showed the most advantageous energy profile (variation of Total EnergyouT/IN of −868.5 kJ mol⁻¹) when the glycosylated extracellular domain was directed outside the vesicle (orientation OUT) compared to its IN orientation (FIG. 3F and FIG. 12). In addition, the Lennard-Jones (LJ) and Coulomb potentials (see FIG. 13A, FIG. 13B, FIG. 14A and FIG. 14B) were calculated to determine the most favorable orientation.

A variation of LJ_{OUT/IN} and Coulomb_{OUT/IN} potentials of −1,041.1 and −36.8 kJ mol⁻¹, respectively, was observed for the glycosylation domain oriented outside the vesicle (OUT) with respect to the inside (IN) orientation (see FIG. 13A, FIG. 13B, FIG. 14A and FIG. 14B). From an energetic standpoint, these findings support the correct orientation of membrane proteins within NA-Leuko bilayer, and suggested that the driving forces of the process were the steric hindrance of the glycosylated domain and the significant reduction of the energetic profile when the glucidic moiety was oriented outside the vesicle.

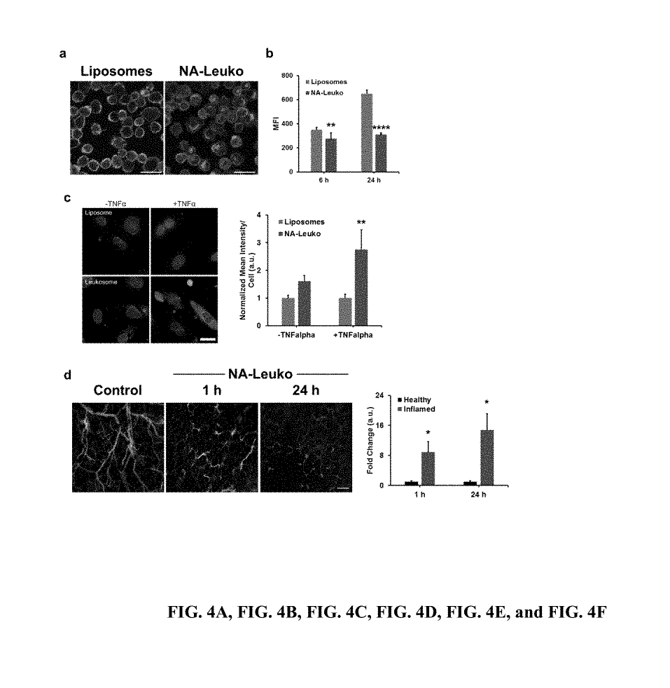

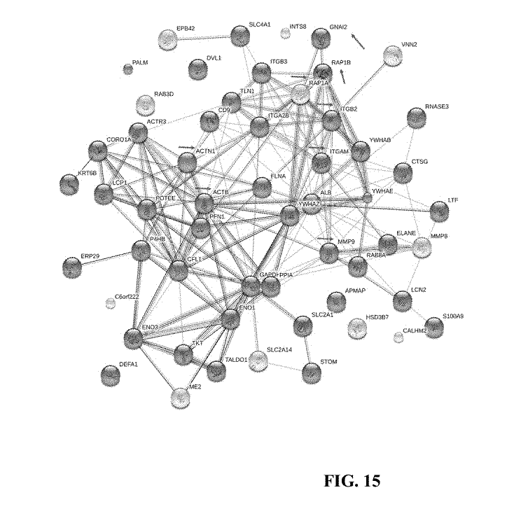

It has been previously shown^{[3b, 7]} that the physical transfer of membrane proteins to synthetic carriers results in the acquisition of novel biological functions.^{[8d, 10]} In this example, it was determined whether the microfluidic-driven synthesis preserved the biological functions of transferred membrane proteins both in vitro and in vivo. The in vitro ability of NA-Leuko incorporating macrophage-derived membrane proteins to delay phagocytosis was determined when incubated with syngeneic macrophages. Compared to control liposomes, a significantly reduced uptake for NA-Leuko was observed at 6 and 24 hr using both confocal microscopy and flow cytometry (FIG. 4A and FIG. 4B). Next, it was determined whether the microfluidic-approach had an effect on the adhesion of human leukocyte membrane proteins towards human umbilical vein endothelial cells (HUVEC), a biological function that was conserved for TLE-Leuko.^{[3b]} Proteomic analysis on human NA-Leuko revealed the presence of adhesion markers responsible for NA-Leuko targeting of inflamed endothelium and identified the leukocyte transedothelial migration as the best represented pathway (FIG. 15). When incubated in flow conditions with a reconstructed monolayer of TNFα-activated HUVEC, NA-Leuko showed a 3-fold increased adhesion compared to control liposomes (FIG. 4B). No statistically-significant difference in the adhesion between control liposomes and NA-Leuko groups was observed on resting (i.e., not inflamed) HUVECs (FIG. 4C), thus highlighting the selectivity of NA-Leuko targeting. To evaluate if this selective inflamed targeting was conserved during systemic administration in mice, and to have a direct comparison with the biological activity of TLE-Leuko, a localized inflammation model was created by subcutaneously injecting lipopolysaccharide (LPS) into the right ear of Balb/c mice, and used the contralateral ear as an internal control permitting inspection of the selective binding of NA-Leuko to inflamed vasculature. While control liposomes did not exhibit any preferential targeting towards inflammation with no difference in accumulation observed between the inflamed and control ear,^{[3b]} NA-Leuko preferentially targeted the inflamed ear at both 1 and 24 hr demonstrating an increase from 8 to 13-fold in accumulation compared to non-inflamed ears, respectively (FIG. 4D). In addition, targeting properties of NA-Leuko were retained over time and could translate to similar drug delivery features as TLE-Leuko. Taken together, these findings revealed the crucial contribution of membrane proteins to the biological properties of these biomimetic nanovesicles in terms of both delay of macrophage uptake, and targeting of inflamed endothelium.

Biomimetic nanoparticles represent promising new generation of drug delivery systems^{[26]}. In this scenario, assembly methods shifted from the manipulation of the whole cell membrane,^{[5]} to its fragmentation in patches^{[3a]} or single membrane proteins^{[3b]}, up to the formulation of hybrid particles by combining cell membranes from two different cells.^{[27]} However, despite several attempts to establish an assembly method capable of formulating clinical-grade nanoparticles, the scale up manufacturing process still represents a major challenge for their clinical translation.^{[28]} The approach we report herein meets this increasing interest in the field and provides a promising tool for overcoming those limitations. Microfluidic platforms offer a high-throughput, low-cost, and scalable tool for the design and production of nanoparticles in a reproducible and standardizable fashion.^{[14]} For the first time, the microfluidic-based NanoAssemblr™ platform has successfully been adapted for the reproducible, robust and versatile synthesis of biomimetic nanoparticles.

The use of this platform includes several advantages such as:

i) accessibility and ease of use;

ii) extensive validation with respect to batch-to-batch reproducibility;

iii) automation, which allows for an easy transfer of synthetic protocol; and

iv) scalability which allows the manufacturing of nanoparticles under cGMP conditions. The resulting formulation revealed suitable pharmaceutical features, i.e., high size homogeneity, unilamellarity, as well as physical stability both at shelf-life and body temperature conditions. Membrane proteins derived from leukocytes were successfully incorporated in their correct orientation and glycosylated, post-translational status. In silico conformational analysis supported our hypothesis that glycosylation sites are responsible for the correct protein orientation within the lipid bilayer of nanovesicles. In addition, in vitro and in vivo biological analyses revealed how NA manufacturing protocols did not affect the function of the key membrane proteins, permitting the avoidance of macrophage uptake and promoting the adhesion to inflamed endothelium. In particular, compared to the previous TLE method,^{[3b]} the NA procedure allowed for 14-fold increase of protein concentration on the surface of Leukosomes, enabling a 1.6-fold increase in vivo accumulation of NA-Leuko at the site of inflammation. Taken together, these findings support the use of this system for the scalability of biomimetic nanovesicles, thus reducing manufacturing-related costs while increasing yield and consistency.

### REFERENCES

The following references, to the extent that they provide exemplary procedural or other details supplementary to those set forth herein, are specifically incorporated herein by reference:
[1] GEBESHUBER, I C et al., “Nanomedicine and biomimetics: life sciences meet engineering & physics,” in ideas, Vol. 6-7 (2009).
[2a] MEYER, R A et al., “Biomimetic particles as therapeutics,” Trends Biotechnol., 33:514-524 (2015).
[2b] BLANCO, E et al., “Principles of nanoparticle design for overcoming biological barriers to drug delivery,” Nature Biotechnol., 33:941-951 (2015).
[2c] A PARODI et al., Biomaterials, 147:155 (2017).
[2d] R MOLINARO et al., Current Medicinal Chem., 24:1 (2017).
[3a] A PARODI et al., Nat. Nanotechnol., 8:61 (2013).
[3b] R MOLINARO et al., Nat. Mater., (2016).
[4] HU, C-M J et al., “Erythrocyte membrane-camouflaged polymeric nanoparticles as a biomimetic delivery platform,” Proc. Nat'l. Acad. Sci. USA, 108:10980-10985 (2011).
[5] HU, C-M J et al., “Nanoparticle biointerfacing by platelet membrane cloaking,” Nature, 526:118-121 (September 2015).
[6] FANG, R H et al., “Cancer cell membrane-coated nanoparticles for anticancer vaccination and drug delivery,” Nano Lett., 14:2181-2188 (2014).
[7] HU, C-M J et al., “A biomimetic nanosponge that absorbs pore-forming toxins,” Nature Nanotechnol., 8:336-340 (2013).
[8a] C CORBO et al., Curr. Drug Targets, 16:1540 (2015).
[8b] M EVANGELOPOULOS et al., Biomaterials, 82:168 (2016).
[8c] R PALOMBA et al., Sci. Rep., 6:34422 (2016).
[8d] C CORBO et al., Nanoscale, (2017).
[9a] C CORBO et al., Biomater. Sci., (2017).
[9b] C CORBO et al., Nanomedicine, 11:81 (2016).
[9c] C CORBO et al., Int. J. Nanomed., 11:3049 (2016).
[9d] M P MONOPOLI et al., Nat. Nanotechnol., 7:779 (2012).
[9e] A SALVATI et al., Nat. Nanotechnol., 8:137 (2013).
[10] C CORBO et al., ACS Nano., 11:3262 (2017).
[11] VALENCIA, P M et al., “Microfluidic technologies for accelerating the clinical translation of nanoparticles,” Nature Nanotechnol., 7:623-629 (2012).
[12] CAPRETTO, L et al., “Microfluidic and lab-on-a-chip preparation routes for organic nanoparticles and vesicular systems for nanomedicine applications,” Adv. Drug Del. Rev., 65:1496-1532 (2013).
[13] JAHN, A et al., “Controlled vesicle self-assembly in microfluidic channels with hydrodynamic focusing,” J. Am. Chem. Society, 126:2674-2675 (2004).
[14] LIU, J et al., “Turbiscan Lab® Expert analysis of the biological demulsification of a water-in-oil emulsion by two biodemulsifiers,” J. Haz. Mat., 190:214-221 (2011).
[15] JAHN, A et al., “Preparation of nanoparticles by continuous-flow microfluidics,” J. Nanopart. Res., 10:925-934 (2008).
[16a] KASTNER, E et al., “High-throughput manufacturing of size-tuned liposomes by a new microfluidics method using enhanced statistical tools for characterization,” Int. J. Pharmaceut., 477:361-368 (2014).
[16b] KASTNER, E et al., “Microfluidic-controlled manufacture of liposomes for the solubilisation of a poorly water soluble drug,” Int. J. Pharmaceut., 485:122-130 (2015).
[16c] CORREIA, M G S et al., “Microfluidic manufacturing of phospholipid nanoparticles: stability, encapsulation efficacy, and drug release,” Int. J. Pharmaceut., (2016).
[17] S GARG et al., J. Drug Target., 24:821 (2016).
[18] AD STROOCK et al., Science, 295:647 (2002).
[19a] PATTNI, B S et al., “New developments in liposomal drug delivery,” Chem. Rev., 115:10938-10966 (2015).
[19b] PERRIE, Y et al., “Manufacturing Methods for Liposome Adjuvants. Vaccine Adjuvants: Methods and Protocols, 2016:127-144 (2016).
[19c] WALSH, C et al., “Microfluidic-based manufacture of siRNA-lipid nanoparticles for therapeutic applications,” Drug Del. Syst., 2014:109-120 (2014).
[20] D LIU et al., Adv. Mater., 27:2298 (2015).
[21] COSCO, D et al., “Aqueous-core PEG-coated PLA nanocapsules for an efficient entrapment of water soluble anticancer drugs and a smart therapeutic response,” Eur. J. Pharmaceut. Biopharmaceut., 89:30-39 (2015).
[22] MARIANECCI, C et al., “Ammonium glycyrrhizinate-loaded niosomes as a potential nanotherapeutic system for anti-inflammatory activity in murine models,” Int. J. Nanomed., 9:635 (2014).
[23a] CELIA, C et al., “Ethosomes® and Transfersomes® containing linoleic acid: physicochemical and technological features of topical drug delivery carriers for the potential treatment of melasma disorders,” Biomed. Microdevices, 14:119-130 (2012).
[23b] J LIU et al., Hazard. Mater., 190:214 (2011).
[23c] MARIANECCI, C et al., “Non-ionic surfactant vesicles in pulmonary glucocorticoid delivery: characterization and interaction with human lung fibroblasts,” Contr. Rel., 147:127-135 (2010).
[24] LUK, B T and ZHANG, L, “Cell membrane-camouflaged nanoparticles for drug delivery,” Contr. Rel., 220:600-607 (2015).
[25] A MAY et al., Bioinformatics, 30:326 (2014).
[26] M GAGLIARDI, Ther. Deliv., 8:289 (2017).
[27] D DEHAINI et al., Adv. Mater., (2017).
[28] S SVENSON, Curr. Opin. Solid State Mater. Sci., 16:287 (2012).
[29] P K DAGUR and J P MCCOY, Curr. Prot. Cytometry, 5.1. 1 (2015).
[30a] SHERMAN, M B et al., “Removal of divalent cations induces structural transitions in red clover necrotic mosaic virus, revealing a potential mechanism for RNA release,” J. Virol., 80:10395-10406 (2006).
[30b] MOLINARO, R et al., “Biomimetic proteolipid vesicles for targeting inflamed tissues,” Nature Materials, (2016).
[31] PARODI, A et al., “Synthetic nanoparticles functionalized with biomimetic leukocyte membranes possess cell-like functions,” Nature Nanotechnol., 8:61-68 (2013).
[32a] RAMACHANDRAN, S et al., “Cisplatin nanoliposomes for cancer therapy: AFM and fluorescence imaging of cisplatin encapsulation, stability, cellular uptake, and toxicity,” Langmuir, 22:8156-8162 (2006).
[32b] MINARDI, S et al., “Evaluation of the osteoinductive potential of a bio-inspired scaffold mimicking the osteogenic niche, for bone augmentation,” Biomaterials, (2015).
[33] D COSCO et al., Eur. J. Pharm. Biopharm., 89:30 (2015).
[34] A ACHOUR et al., Immunity, 9:199 (1998).
[35] Y Q I et al., J. Chem. Theory Comput., 11:4486 (2015).
[36] S J O et al., J. Comput. Chem., 29:1859 (2008).
[37a] L MONTICELLI et al., J. Chem. Theory Comput., 4:819 (2008).
[37b] S-J MARRINK et al., J. Phys. Chem. B, 111:7812 (2007).
[38] D H DE JONG et al., J. Chem. Theory Comput., 9:687 (2012).
[39] H J BERENDSEN et al., J. Chem. Phys., 81:3684 (1984).
[40] G BUSSI et al., J. Chem. Phys., 126:014101 (2007).

It should be understood that the examples and embodiments described herein are for illustrative purposes only and that various modifications or changes in light thereof will be suggested to persons skilled in the art and are to be included within the spirit and purview of this application and the scope of the appended claims. All references, including publications, patent applications and patents, cited herein are specifically incorporated herein by reference to the same extent as if each reference was individually and specifically indicated to be incorporated by reference, and was set forth in its entirety herein. Recitation of ranges of values herein are merely intended to serve as a shorthand method of referring individually to each separate value falling within the range, unless otherwise indicated herein, and each separate value is incorporated into the specification as if it were individually recited herein.

The description herein of any aspect or embodiment of the invention using terms such as “comprising,” “having,” “including,” or “containing,” with reference to an element or elements is intended to provide support for a similar aspect or embodiment of the invention that “consists of,” “consists essentially of,” or “substantially comprises” the particular element or elements, unless otherwise stated or clearly contradicted by context (e.g., a composition described herein as comprising a particular element should be understood as also describing a composition consisting of that element, unless otherwise stated or clearly contradicted by context).

All of the compositions and methods disclosed and claimed herein can be made and executed without undue experimentation in light of the present disclosure. While the compositions and methods of this invention have been described in terms of preferred embodiments, it will be apparent to those of ordinary skill in the art that variations may be applied to the compositions and methods and in the steps or in the sequence of steps of the method described herein without departing from the concept, spirit and scope of the invention. More specifically, it will be apparent that certain agents that are chemically- or physiologically-related may be substituted for the agents described herein while the same or similar results would be achieved. All such similar substitutes and modifications apparent to those ordinarily skilled in the art are deemed to be within the spirit, scope, and concept of the invention as defined by the appended claims.

What is claimed is:

1. A method of preparing a population of biomimetic proteolipid nanovesicles composed of synthetic phospholipids and cholesterol, enriched of leukocyte membrane fragments, and surrounding an aqueous core, the method comprising:
a) dissolving predetermined amounts of two or more selected phosphocholine-based phospholipids, and cholesterol in ethanol to a final lipid concentration of approximately 9 mM to produce an organic lipid solution;
b) dissolving a predetermined amount of at least one selected membrane protein in water to produce an aqueous protein solution;
c) loading the organic lipid solution of (a) into the organic phase inlet of a microfluidic mixer, and loading the aqueous protein solution of (b) into the aqueous phase inlet of the microfluidic mixer, wherein the microfluidic mixer is set to a predetermined reaction temperature; and
d) adjusting the flow rates of each inlet stream and the flow ratio between each of the inlet streams of the microfluidic mixer to produce the population of biomimetic proteolipid nanovesicles therefrom.

2. The method of claim 1, wherein adjustment of the flow rates or the flow ratio results in the production of a population of biomimetic proteolipid nanovesicles having a desired property selected from the group consisting of a consistent size, a consistent size homogeneity, a selected amount of protein incorporation into the membrane fragments, nanovesicle stability, and any combination thereof.

3. The method of claim 1, further comprising purifying the population of biomimetic proteolipid nanovesicles so produced by dialysis, ultracentrifugation, or a combination thereof.

4. The method of claim 1, wherein the selected flow rate is 1 mL/min, the selected flow ratio is 2:1 (organic phase-to-aqueous phase), and the predetermined reaction temperature is approximately 45° C.

5. The method of claim 5, wherein the efficiency of protein incorporation into the leukocyte membrane fragments is at least 40% to 60% higher than that of biosimilar nanovesicles prepared by conventional thin-layer evaporation.

6. The method of claim 1, wherein the total number of nanovesicles produced per gram of lipid is at least 100% higher than that of biosimilar nanovesicles prepared by conventional thin-layer evaporation.

7. The method of claim 6, wherein the total number of nanovesicles produced per gram of lipid is at least 200% higher than that of biosimilar nanovesicles prepared by conventional thin-layer evaporation.

8. The method of claim 1, wherein the efficiency of protein incorporation into the membrane fragments is at least 10-fold higher than for biosimilar nanovesicles prepared by conventional thin-layer evaporation.

9. The method of claim 8, wherein the efficiency of protein incorporation into the membrane fragments is at least 20-fold higher than for biosimilar nanovesicles prepared by conventional thin-layer evaporation.

10. The method of claim 1, wherein the proteolipid nanovesicles are about 100 to about 1000 nm in average diameter.

11. The method of claim 1, wherein the selected phosphocholine-based phospholipids are selected from the group consisting of phosphatidylcholine, egg phosphatidic acid, 1,2-dioleoyl-sn-glycerophosphocholine (DOPC), 1,2-dioleoyl-sn-glycerophosphoethanolamine (DOPE), 1,2-dipalmitoyl-sn-glycerophosphocholine (DPPC), 1,2-distearoyl-sn-glycerophosphocholine (DSPC), and any combination thereof.

12. The method of claim 1, wherein the resulting population of biomimetic proteolipid nanovesicles comprises substantially spherical, unilamellar vesicles.

13. The method of claim 1, wherein the resulting population of biomimetic proteolipid nanovesicles are stable in solution at 4° C. for about two to about three weeks.

14. The method of claim 1, wherein the resulting population of biomimetic proteolipid nanovesicles are stable in solution at 4° C. for at least 24 days after synthesis, without significant change in either average nanovesicle diameter or particle size homogeneity.

15. The method of claim 1, wherein the proteolipid nanovesicles are from about 100 mm to about 1000 nm in average diameter.

16. The method of claim 15, wherein the proteolipid nanovesicles are from about 200 mm to about 600 nm in average diameter.

17. The method of claim 1, wherein the protein-to-lipid ratio is from about 1:50 (wt./wt.) to about 1:500 (wt./wt.).

18. The method of claim 17, wherein the protein-to-lipid ratio is from about 1:100 (wt./wt.) to about 1:300 (wt./wt.).

19. The method of claim 1, wherein the resulting population of biomimetic proteolipid nanovesicles further comprises a diagnostic or therapeutic agent, or a combination thereof.

20. The method of claim 19, wherein the therapeutic agent comprises an siRNA that is specific for a mammalian gene selected from the group consisting of BRAF, MEK, ERK1, and ERK2.

21. A population of biomimetic proteolipid nanovesicles prepared by the method of claim 1.

22. A drug delivery composition comprising the population of biomimetic proteolipid nanovesicles of claim 21.

23. The drug delivery composition of claim 22, wherein the proteolipid nanovesicles comprise at least one self-tolerance protein or active fragment thereof on their surface selected from the group consisting of LFA-1, CD47, CD-45, CD-47, and MHC-1.

24. The drug delivery composition of claim 22, further comprising at least one therapeutic agent, including, for example, a chemotherapeutic drug, an antibiotic, an analgesic, or a siRNA molecule.

25. The drug delivery composition of claim 22, wherein the leukocyte membrane fragments are derived from human leukocyte plasma membranes.

26. The drug delivery composition of claim 22, further comprising at least one therapeutic agent selected from the group consisting of an immune-stimulating agent, a tumor growth inhibitor, a protein, a peptide, an RNA molecule, a DNA molecule, an siRNA molecule, a RNAi molecule, a ssRNA molecule, a growth factor, an enzyme inhibitor, a binding protein, a blocking peptide, and any combination thereof.

27. The drug delivery composition of claim 22, wherein the proteolipid nanovesicles are adapted configured to release the at least one therapeutic agent in response to an external stimulus, in response to a change in the environment of the population of biomimetic proteolipid nanovesicles, or as a result of degradation of the proteolipid nanovesicles.

28. The drug delivery composition of claim 22, wherein degradation of the population of biomimetic proteolipid nanovesicles occurs via enzyme-facilitated biodegradation of one or more of the phospholipids or the cholesterol comprising them.

29. The drug delivery composition of claim 22, wherein the leukosyte membrane fragments comprise at least one cellular-targeting moiety.

30. The drug delivery composition of claim 29, wherein the at least one cellular-targeting moiety is selected from the group consisting of a chemically-targeting moiety, a physically-targeting moiety, a geometrically-targeting moiety, a ligand, a ligand-binding moiety, a receptor, a receptor-binding moiety, an antibody or an antigen-binding fragment thereof, and any combination thereof.

31. The drug delivery composition of claim 30, wherein the at least a first cellular-targeting moiety comprises a plurality of distinct antigenic ligands that elicit one or more target-specific immune responses in a mammalian host cell that is contacted with the population of nanovesicles.

32. The drug delivery composition of claim 22, further comprising a diagnostic agent.

33. The drug delivery composition of claim 32, wherein the diagnostic reagent is selected from the group consisting of an imaging agent, a contrast agent, a fluorescent label, a radiolabel, a magnetic resonance imaging label, a spin label, and any combination thereof.

34. The drug delivery composition of claim 22, comprising a chemically-targeting moiety that is disposed on the surface of the proteolipid nanovesicles, and that comprises a ligand, a dendrimer, an oligomer, an aptamer, a binding protein, an antibody, an antigen-binding fragment thereof, a biomolecule, or any combination thereof.

35. A population of isolated mammalian cells comprising the population of biomimetic proteolipid nanovesicles of claim 21.

36. A pharmaceutical formulation comprising the population of biomimetic proteolipid nanovesicles of claim 21, and a pharmaceutically-acceptable buffer, diluent, excipient, or vehicle.

37. A kit comprising the pharmaceutical formulation of claim 36, and instructions for administering the composition to a mammal in need thereof, as part of a regimen for the prevention, diagnosis, treatment, or amelioration of one or more symptoms of a disease, a dysfunction, an abnormal condition, or a trauma in the mammal.

38. A method for providing one or more active agents to a population of cells within the body of an animal, comprising administering to the animal an amount of the pharmaceutical formulation of claim 36, for a time effective to provide the one or more active agents to the population of cells within the body of the animal.

39. The method of claim 38, wherein the animal is at risk for developing, is suspected of having, or is diagnosed with a tumor or a cancer.

40. A method of targeting a diagnostic, therapeutic, or prophylactic agent to one or more inflamed sites within the body of a mammalian subject in need thereof, comprising administering to the subject an effective amount of the pharmaceutical formulation of claim 36.

41. The method of claim 40, wherein the therapeutic agent comprises at least a first siRNA, DNA, ssRNA, RNAi, or any combination thereof.

42. The method of claim 40, wherein the therapeutic agent further comprises at least a first chemotherapeutic agent.

43. The method of claim 40, wherein accumulation of the biomimetic proteolipid nanovesicles is at least 8- to 13-fold higher in the inflamed site as compared to non-inflamed tissues when the formulation is administered systemically to the mammal.
